(19) United States
(12) Patent Application Publication
(10) Pub. No.: US 2026/0357265 A1
(43) Pub. Date: Nov. 12, 2026

(54) ANTI-VIRAL SIRNA THERAPY

(71) Applicant: TECHNISCHE UNIVERSITÄT BERLIN, Berlin (DE)

(72) Inventors: Henry FECHNER, Luckau (DE); Anja GEISLER, Bernau (DE)

(21) Appl. No.: 19/471,593

(22) PCT Filed: Mar. 20, 2024
(86) PCT No.: PCT/EP2024/057414

§ 371 Date: Oct. 1, 2025

(30) Foreign Application Priority Data
Apr. 3, 2023 (DE) 10 2023 108 413.5

(51) Int. Cl. C12N 15/113; A61P 31/20

(52) U.S. Cl. CPC C12N 15/1131; A61P 31/20; C12N 2310/14; C12N 2310/315; C12N 2310/321

(57) ABSTRACT

The invention refers to modified anti-viral siRNA, encapsulated in e.g. lipid nanoparticles (LNP) for treatment of viral infections, and in particular treating infections caused by human adenovirus (hAd).

### REFERENCE TO SEQUENCE LISTING

The instant application contains a Sequence Listing which has been submitted electronically in XML format and is hereby incorporated by reference in its entirety. Said XML copy, created on Jul. 16, 2026, is named “50322-011001_Sequence_Listing_7_16_26” and is 86,796 bytes in size.

### FIELD OF THE INVENTION

The invention refers to modified anti-viral siRNA, encapsulated in lipid nanoparticles (LNP) for treatment of viral infections, and in particular treating infections caused by human adenovirus (hAd).

### BACKGROUND OF THE INVENTION

As up to now there is no proper treatment for viral infections in general, and particularly human adenoviruses cause regularly untreatable but highly infectious outbreaks.

The human adenovirus (hAd) can infect humans of any age, young children and infants are most often affected. Due to genetic heterogeneity resulting in different tissue tropisms, hAd causes various organ infections, mainly affecting the respiratory tract, the eyes and the intestine, but infections of the genitourinary tract, the heart, the brain and the liver have also been observed. In patients with an intact immune system, these infections are generally acute and self-limiting, with mild symptoms, which is why they are usually treated symptomatically. In contrast to this course of infection, in immunocompromised patients hAd can induce severe disease, for example in patients with congenital immunodeficiency or after infection with the human immunodeficiency virus, or in children receiving chemotherapy for hematological cancer diseases and in solid organ transplant recipients. Allogeneic hematopoietic stem cell transplantation (HSCT) patients, usually young children, represent a group with a particularly high risk for life-threatening infection by hAd. For example, Fisher et al. found that among 191 allogeneic HSCT recipients, 58 (30.4%) were infected with hAd. Fifteen of the patients died and two-thirds of these deaths were related to the progression of hAd disease. Causes of death include multiorgan failure due to disseminated hAd infection or liver failure due to massive hAd replication and liver tissue damage.

Human adenovirus, hAd, belong to the family Adenoviridae within the genus Mastadenovirus and can be divided into seven species (A-G), made up of more than 100 different types. Adenoviruses are medium-sized (90-100 nm), non-enveloped, double-stranded DNA viruses with an icosahedral nucleocapsid, which genome has a length of 35 kb. Adenoviruses are widespread in vertebrate hosts, but at the same time are highly species specific. They are responsible for multiple illnesses including respiratory infections, conjunctivitis, gastroenteritis, probably also obesity and multiple-organ diseases in immune compromised patients.

There is no FDA- or EMA-approved antiviral therapy for the treatment of any hAd infections.

As a result, the only therapy options are off-label use of drugs approved for other indications. Severe hAd infections are most commonly treated with the nucleotide phosphate cytosine analog cidofovir (CDV) and the orally bioavailable lipid-linked derivative of CDV, brincidofovir (BCV), whereas ribavirin and ganciclovir were used less frequently. However, only a small majority of patients profit from the treatment. Moreover, CDV is nephrotoxic and gastrointestinal toxicity upon oral application in a phase III clinical trial was observed after treatment of patients with BCV. Another promising therapy for such infections is hAd-specific T-cell therapy, but because of its personalized nature, this therapy is costly and time-consuming, thereby limiting its application in patients.

About a decade ago it was shown that hAd infections can be efficiently inhibited in vitro with anti-adenoviral siRNAs, which induces post-transcriptional gene silencing via conserved cellular mechanism of RNA interference (RNAi). The nature of adenoviral infection and viral replication defined several adenoviral proteins as potential targets of therapeutic RNAi and indeed it has been confirmed that hAd infection can be inhibited in vitro by silencing the adenoviral protein. Inhibition of adenoviral replication by RNAi was achieved by targeting both pTP and Pol. Both proteins play a central role in the replication of the adenoviral genome, indicating that a disturbance of the adenoviral DNA replication machinery represents a potent approach to inhibit hAd infections.

Despite firsts promising results, the use of siRNA therapeutics is still not successfully introduced into therapeutic approaches and is restricted or hampered by e.g. its off-target toxicity, a limited efficacy due to innate immune activation, a substantial lack of stability against RNA degradation and/or inefficient delivery to the target tissue.

It is thus, an objective of the present invention to improve siRNA for the therapeutic use and particularly, to develop siRNA for use in a treatment of viral infections in vivo.

Beside others, this object is solved by the present invention in providing a modified siRNA according to claim 1. Further advantageous details, aspects and embodiments of the present invention are represented by the embodiments of the dependent claims.

In one embodiment due to the efforts of the inventors a modified siRNA molecule is provided, which proofed to be highly effective in the treatment of viral infections, and particularly the inhibition of adenovirus infection.

In particular the invention provides a modified siRNA consisting of a guide and a passenger strand, wherein the guide strand is subdivided into a 5′-region, a complementary region to a viral target sequence, and a 3′-region, and wherein the guide strand is characterized in that said 5′-region comprises at least one deoxyribose nucleotide, said complementary region comprises a least one 2′-ribose modification, and said 3′-region comprises at least one phosphate backbone modification and optionally one or more 2′-ribose modifications.

In the context of the present invention the term “small interfering RNA (siRNA)” refers to a defined single or double stranded RNA sequence, which can trigger the cellular defense mechanism of RNA interference (RNAi). siRNA does not require to recognize complex spatial conformations of proteins, because its mode of action is based on a highly specific base pairing between nucleic acids of the siRNA, e.g. with other RNA. siRNA generally comprises short double stranded RNA molecules, more precise a guide strand and a passenger strand. The guide strand directs an RNA-induced silencing complex (RISC) to the target RNA by recognizing and binding a complementary sequence. Without the guide strand no targeting to a specific RNA sequence would be possible, it is thus considered the crucial element for an effective silencing.

In the context of the invention, it was shown that the chosen modifications of the siRNA, particularly in the guide strand, did lead to improvements not losses of the siRNA properties, such as stability and or specificity.

In the context of the present invention the term “guide strand” relates to the complementary (antisense) RNA pairing sequence of the modified siRNA and is inducing the post-transcriptional gene silencing by binding to a target RNA. Further, in the context of the present invention the term “target RNA” refers to a coding or non-coding RNA sequence, which forms the primary target of the modified siRNA of the invention. The sequence of the target RNA can vary in its length but must comprise a sequence segment which allows complementary binding of the guide strand. “Coding sequence” herein refers to an RNA sequence that codes for a specific amino acid sequence, e.g. protein.

The term “nucleotide” relates in the context of this invention to a unit consisting of a five-carbon sugar molecule, a nucleobase-adenine (A), cytosine (C), guanine (G), thymine (T) or uracil (U)- and at least one phosphate group. In a sequence of nucleotides, e.g. DNA or RNA, the phosphate group may be referred to as a phosphate backbone.

According to the teaching of the present invention the modified siRNA comprises chemically modifications which are located at the five-carbon sugar molecule, the nucleobase and/or the phosphate group of a nucleotide to alter the properties to improve stability, activity, and potential off-target effects, especially but not limited for a therapeutic use.

According to the invention the guide strand is, as already mentioned above, subdivided into a 5′-region, a complementary region to a viral target sequence, and a 3′-region. Additionally, the 5′ of the guide strand is characterized by its phosphorylated 5′-end comprising at least one but also two or three deoxyribose nucleotides, without being bound to the hypothesis the inventors believe, that an exchange of one or more of the ribose nucleotides into deoxyribose nucleotides will increase the stability of the siRNA, thus the silencing activity.

Deoxyribose nucleotides according to the invention can be selected from the group comprising deoxyadenosine monophosphate, deoxythymidine monophosphate, deoxycytidine monophosphate and/or deoxyguanosine monophosphate, including the respective di- or triphosphates.

Also, the complementary region to a viral target sequence of the guide strand according to the invention is characterized by a sequence of variable length, typically by a sequence between 16 and 24 ribose nucleotides, preferably with 16, 17, 18, 19, 20, 21, 22, 23 or 24 ribose nucleotides complementary to a viral RNA target sequence. While for ideal sequence matching 80-99% base complementarity is necessary, it was found that the guide strand of the present invention can tolerate 1 or 2 or even up to 4 mismatches on a length of 16-24 ribose nucleotides complementary to the viral target sequence without losing its binding capacity and thus silencing effect.

These around 70-90% complementarity is particularly interesting, as with this capacity to specifically bind viral target sequences although there are 1 to 4 mismatches between the viral target sequence and the modified siRNA of the invention, the specificity to further closely related viruses is increased and thus the effectivity of the silencing effect is extended to related strains and subtypes.

Ribose nucleotides of the complementary region to a viral target sequence in the context of the invention comprises at least 1 but also 2, 3, 4, 5 or more 2′-ribose modifications to increase the binding affinity to a viral target sequence, improve resistance against ribonucleases and/or reduce any unwanted off-target immunogenicity in vivo. Additionally, modifications of further ribose positions for example the 4′-C position or a modification of the whole ring-form can further alter the properties of the siRNA to improve their utilization.

The 3′-region of the guide strand according to the invention is characterized by a hydroxylated 3′-end, comprising 1, 2 or 3 ribose nucleotides and forming an overhang structure to the passenger strand. At least one but also two or up to three ribose nucleotides of the 3′-region comprise a phosphate backbone modification and optionally a 2′-ribose modification. Modifications of the phosphate group improve resistance against ribonucleases, increase the bioavailability, and/or vary the binding affinity to the target RNA. Optional ribose modifications may provide further advantages, as e.g. resistance against nucleases and improved binding affinity.

The inventors found the most effective modification pattern of the guide strand can be described in a generalized structure: 5′-(X/X_{d})₁₋₃-(Y/Yₘ)₁₈₋₂₃-(Z/Zₘ/Z_{b}/Z_{m+b})₁₋₃
where the ribose nucleotides of the sequence are represented by the capital letters X, Y and Z, wherein X stands for the ribose nucleotides of the 5′-region, Y for the ribose nucleotides of the region complementary to a viral target region, and Z for the ribose nucleotides of the 3′-region. The type of modification intended at the various nucleotides positions is indicated by the subscript letter, where m indicates a ribose modification, b indicates a phosphate backbone modification and d indicates the presence of a DNA nucleotide at this position, no subscript indicates an unmodified ribose nucleotide. The slash between e.g. “(X/X_{d})” indicates that any of the listed ribose nucleotides, with or without modification can be selected. Finally, the number outside of the brackets indicates the possible number of unmodified or modified ribose nucleotides and thus the length of the region.

In one embodiment of the invention, the modification pattern of the guide strand corresponds to the following sequence pattern: 5′-X_{d}-Y-Y-Y-Y-Y-Y-Y-Y-Y-Y-Y-Yₘ-Y-Y-Y-Y-Y-Y-Zₘ-Z_{m+b}-3′ Letters and subscripts follow the above description.

Additional to the guide strand, the modified siRNA comprises a passenger strand (corresponds to the sense strand) which can be subdivided into a 3′-region and a region complementary to the guide strand, which can be addressed also as antisense strand. In the context of the invention the term “passenger strand” refers to a complementary pairing sequence to the guide strand.

The 3′-region of the passenger strand according to the invention is characterized by a hydroxylated 3′-end comprising 1, 2 or 3 ribose nucleotides. Further, at least one but also two or up to three ribose nucleotides of the 3′-region comprise a phosphate backbone modification and optionally a 2′-ribose modification.

The complementary region to the guide strand according to the invention is characterized by a phosphorylated 5′-end and a sequence of variable length, typically of a sequence between 16 and 24 ribose nucleotides, preferably with 16, 17, 18, 19, 20, 21, 22, 23, or 24 ribose nucleotides complementary to the guide strand. 40-70% of the ribose nucleotides of the complementary region to the guide strand comprise a 2′-ribose modification.

2′-ribose modifications of nucleotides allow a broader binding affinity to the target sequence and improve the nuclease resistance of the modified siRNA of the present invention. In addition, modifications of the phosphate backbone may provide further improved resistance against degradation and improved bioavailability of the modified siRNA of the present invention.

The inventors also found the most effective modification pattern of the passenger strand can be described as follows: 5′-(V/Vₘ)₁₈₋₂₃-(Z/Zₚ/Z_{m+b})₁₋₃-3′ where the ribose nucleotides of the sequence are represented by the capital letters V and Z, wherein V stands for the ribose nucleotides of the complementary region to the guide strand and Z for the ribose nucleotides of the 3′-region. Similar to the description of the guide strand, the type of modification is indicated by the subscripts. Where m indicates a ribose modification, b indicates a phosphate backbone modification, and no subscript indicates an unmodified RNA nucleotide. The slash between e.g. “(V/Vₘ)” indicates that any of the listed ribose nucleotides, with or without modification, can be selected for the position. The numbers outside of the brackets indicate the possible number of unmodified or modified ribose nucleotides and thus the length of the region.

In one embodiment of the invention, the modification pattern of the passenger strand corresponds to the following sequence pattern: 5′-Vₘ-Vₘ-V-Vₘ-Vₘ-Vₘ-V-V-V-Vₘ-Vₘ-V-Vₘ-Vₘ-Vₘ-Vₘ-V-Vₘ-Z_{m+b}-3′ Letters and subscripts follow the above description.

While unmodified siRNA is typically degraded by nucleases and possesses poor membrane permeation qualities, the modified siRNA of the invention comprising the chemical modifications of the guide and passenger strand as described before do show a significant improvement regarding a reduced nuclease degradation and thereby-without being bound by this hypothesis-most likely a prolonged in vivo stability. Due to the improved stability a prolonged binding and thus, improved silencing activity follows.

While potentially such modifications can be placed at various and different position within a nucleotide, it was found that e.g. modifications of the five-carbon sugar molecule can increase the resistance against hydrolysis by ribonucleases and therefore enhances the stability of the siRNA, especially in vivo. In addition, the results show that with the modifications of the invention the affinity to the target RNA can be increased and the immunogenicity decreased. Thus, the modified siRNA shows fewer side effects but still functions as effective viral therapeutic.

According to further embodiments 2′-C or 4′-C modifications or modifications of the entire sugar ring can be selected from, but are not limited to, the group of 2′-O-methyl(2′-OMe), 2′-O-methoxyethyl(2′-O-MOE), 2′-deoxy-2′-fluoro (2′-F), 2′-arabino-fluoro (2′-Ara-F), 2′-O-benzyl(2′-O-Bn), 2′-O-methyl-4-pyridine (2′-O-CH₂Py(4)), locked nucleic acid (LNA), (S)-cEt-BNA, tricyclo-DNA (tcDNA), morpholino oligonucleotide (PMO), unlocked nucleic acid (UNA) and glycol nucleic acid (GNA).

In the context of the present invention 2′-ribose modifications are preferably selected from the group comprising 2′-O-methyl(2′-OMe), 2′-O-methoyxethyl(2′-O-MOE), 2′-deoxy-2′-fluoro (2′-F), 2′-arabino-fluoro (2′-Ara-F), 2′-O-benzyl(2′-O-Bn) and 2′-O-methyl-4-pyridine (2′-O-CH₂Py(4)).

Further, modifications of the phosphate group can result in more hydrophobic and thus, stable molecules with a higher affinity to plasma proteins, e.g. albumin, to increase the half-life of oligonucleotides in circulation. Suitable phosphate backbone modifications can according to the invention be selected from the group comprising phosphorothioate (PS), phosphorodithioate (PS2), methylphosphonate (MP), methoxypropylphosphonate (MOP), 5′-(E)-vinylphosphonate (5′-(E)-VP), 5′-methyl phosphonate (5′-MP), (S)-5′-C-methyl with phosphonate, 5′-phosphorothioate (5′-PS) and peptide nucleic acid (PNA).

In the context of the invention phosphate backbone modifications are preferably selected from the group comprising phosphorothioate (PS), phosphorodithioate (PS2), methylphosphonate (MP) and methoxypropylphosphonate (MOP).

For these modifications in the modified siRNA of the invention it was shown that an adenovirus infection can effectively be silenced in vivo and thus such viral infection can be successfully treated.

The examples demonstrate also that a hAd infection was successfully treated in the in vivo model of hAd5-induced hepatitis using immunosuppressed Syrian hamsters. In this model, e.g. self-complementary adeno-associated virus (AAV) vectors of the serotype 9 were used as a carrier to deliver anti-adenoviral artificial microRNAs (amiRs) to the liver. Here, a strong inhibition of hepatic hAd infection was observed when such vector was applied two weeks before the animals were infected with hAd5, whereas application of the AAV vectors concomitant with hAd5 infection resulted in very low inhibition of hepatic hAd infection. While these results show that in principle the silencing is a valid approach, however, up to now the delivery of stable silencing RNA into cells infected by a virus is challenging.

With the above-described modifications of the siRNA molecules the inventor had for the first time a siRNA in hands which was stable enough to be used for a therapeutic application in vivo, was suitable to be delivered with the help of various encapsulation techniques to the virus infected organs and cells, and was stable enough to effectively silence the replication of the hAd after successful delivery in the virus infected organ or the virus infected cells and although being a matching target sequence for one hAd, the siRNA is useful and safe to treat multiple different subtypes of hAd even with one or several mismatches in the corresponding target sequence.

Accordingly, the invention provides the modified siRNA according to the invention for use in treatment and/or prevention of a virus infection in humans, animals and/or plants.

In one preferred embodiment the modified siRNA according to the invention targets and silences the translation of viral proteins of the family Adenoviridae, particularly Adenoviridae within the genus Mastadenovirus.

Adenoviruses or Mastadenoviruses are responsible for various human illnesses and diseases. Related symptoms of such adenoviral diseases comprising hepatitis, gastroenteritis, keratoconjunctivitis, cystitis, rhinitis, pharyngitis, diarrhea, respiratory diseases, and obesity. A further serious thread are adenoviral infections for immunocompromised patients and patients which have received an organ transplantation.

For a successful treatment of a viral infection with a modified siRNA according to the invention, it is preferred to target viral sequence, which may be selected from coding or non-coding mRNA of the relevant virus. Particularly interesting in this context are coding sequences of structural and non-structural viral proteins, which, when silenced, have a seriously hampering effect on the virus replication of the selected target virus.

In the context of the invention, the viral target sequence may vary in their length, typically between 16 and 24 nucleotides, preferably the target sequence consists of a length of 16, 17, 18, 19, 20, 21, 22, 23 or 24 nucleotides. Extension of the target region and therefore also extension of the modified siRNA may increase the binding affinity and also the number of tolerated mismatches. However, such elongated siRNA molecules do need high end encapsulation for assisting the stability and the transportation to the virus infected target cells. According to further embodiments the viral target sequence may thus be prolonged by up to 10 nucleotides.

As it is known that mismatches between the guide strand and the RNA target sequence of the virus may occur for example due to mutations or alternation in the sequence of various subtypes of the virus, such prolongations and thus the possibility to increase the binding activity are a further improvement for the modified siRNA according to the invention.

According to a preferred embodiment the “targeted viral sequence” derives from an Adenovirus. It is well-known that the genes of adenoviruses can generally be divided into well-conserved sets of transcription units with six early transcription units (E1A, E1B, E2A, E2B, E3 and E4) and one late transcription unit ranging from L1-L5. In addition, adenoviruses also contain two intermediate transcription units named XI and IVa2. To increase the viral gene economy, adenoviruses accommodate genes on both strands of its dsDNA meaning that most of its genome is utilized for coding proteins.

The present invention has demonstrated its effectiveness for some of the adenoviral proteins but is not to be understood as a limitation. As shown in the examples as targeted viral coding sequences have been herein used some of the following coding sequences: DNA Polymerase, pre-terminal protein, IVa2, E1A and/or the hexon protein.

In further embodiments of the present invention, the complementary region to the viral target sequence in the guide strand of the modified siRNA is selected from the group of SEQ ID: No 39, SEQ ID: No 41 and SEQ ID: No 43.

While the present disclosure shows the effectiveness of the claimed siRNA and particularly, the effectiveness of the in vivo therapy regarding adenoviral infections, the selection of viral target sequence may be chosen depending on the adenoviral subtype and/or the relevant adenoviral-caused symptoms to be treated.

The adenoviral life cycle is divided in an early and a late phase. While in the early phase mainly non-structural and regulatory proteins are expressed, which cause the infected cell to hide from host-immune defense strategies, by e.g. blockage of interferon activity or MHC class I expression, to avoid premature cell death and to prepare for viral protein synthesis, the late phase is dominated by an active replication of virus genome sequences and structural proteins. Accordingly, the choice of the viral target sequence to be silenced with the modified siRNA of the invention depends on the illness and will in case of a quickly spreading conjunctivitis focus on early expressed regulatory targets together with e.g. the viral DNA polymerase, which when silenced, will effectively avoid further replication. On the other hand, for patients with more chronic adenoviral illnesses a double strategy targeting and silencing structural proteins, which are expressed later in the life cycle may proof advantageous.

In any case, the modified siRNA molecule of the present invention, even if the stability is improved, needs to be additionally protected for the transport to the target organ and/or into the virus infected cell.

Recent years have increased the knowledge of suitable delivery systems for oligonucleotides, which have proven to be beneficial in overcoming biological barriers in vivo and to transport their pharmacological or therapeutical cargo more effective to a target cell. Known delivery systems can be divided into the group of chemical conjugates or nanoparticle carriers.

Lipid nanoparticles (LNP) as one representative of such nanoparticle carrier are a non-viral lipid vesicle with a homogenous lipid core. They are one of the most used systems for delivery of small-molecule drugs and nucleic acids. In the last few years LNP-based delivery of nucleic acids has received a great deal of attention, as it has been used as a delivery platform for COVID19-mRNA vaccines Spikevax and Comirnaty and an FDA-approved LNP-siRNA for treatment of the hereditary transthyretin amyloidosis. LNP are composed of different components such as ionizable lipids, cationic lipids, structural lipids (cholesterol and phospholipids) and poly(ethylene glycol)-anchored lipids to aid in covering and protecting the nucleic acids to be transported, and to aid in passing through the cell and nuclear membranes.

The internalization and degradation of LNP, their recognition by the immune system, their duration in the blood circulation and their biological distribution are affected by proteins surrounding the out-layer of LNP, as well as by the size and charge of the LNP. Since systemic application leads to efficient perfusion of the liver, LNP are herein used for delivery of siRNAs into hepatocytes. Once in the bloodstream, LNP-encapsulated siRNAs (LNP-siRNA) bind to serum proteins, including apolipoprotein E, which in turn binds to the low-density lipoprotein receptor. This receptor is highly expressed on hepatocytes and directs the LNP-siRNA into these cells.

Nanoparticle carriers are used to encapsulate the modified siRNA of the invention and thereby additionally improving the protection against degradation and/or enhancing the targeting. Suitable nanoparticle carriers are selected from the group of lipid-, polymer- and peptide-based delivery systems as well as hybrids of these.

Consequently, the term “encapsulated” in the context of the present invention refers to the modified siRNA of the invention fully encapsulated and/or partially encapsuled in a lipid nanoparticle material, wherein the lipid nanoparticle material is selected from the group comprising cationic lipids, ionizable lipids, structural lipids, and glycol anchored lipids.

In some experiments it was shown that much of the LNP-siRNA is taken up by Kupffer cells, sinusoidal endothelial cells and stellate cells, resulting in a significant amount of siRNA not accumulating in hepatocytes. In case of an approach for targeting hepatocytes only, the latter can be largely avoided by changing the lipid composition, in the present case to a LNP-siRNAs using appropriate cationic amino-lipids.

In a further embodiment of the present invention, the lipid nanoparticle material for encapsulating the modified siRNA comprises the cationic amino-lipid XL-10.

Alternatively, also chemical conjugates may be used to encapsulate and/or protect the modified siRNA of the invention by forming a covalent bond formation of a delivery system to the siRNA and thus, favorably also alter the properties in terms of toxicity, target specificity, and bioavailability. Suitable molecules used for conjugation can be selected from the group comprising polymers, peptides, lipids, receptor ligands, antibodies, and aptamers. Especially, receptor ligands can be used to increase a tissue specific active targeting, to improve the efficiency of the modified siRNA.

Interestingly, according to a further embodiment of the invention, also N-acetylgalactosamine (GalNAc) ligand linked to the 5′-end of modified siRNA can be used to protect the siRNA of the present invention and additionally, such N-acetylgalactosamine ligands are also suitable to improve the target specificity in hepatocytes.

In this embodiment of the present invention, the chemical conjugate for protecting the modified siRNA is a triantennary GalNAc ligand, which is known to bind to the Asialglycoprotein receptors on hepatocytes. The modified siRNA linked to the triantennary GalNAc ligand can thus enter the cytoplasm of hepatocytes more effectively and induce a stronger target specific RNAi response.

For medical uses of the invention and administration to patients the above-described modified siRNAs are provided in form of a pharmaceutical composition comprising an effective amount of the modified siRNA, be it pure, encapsulated or protected, and a suitable additive such as a pharmaceutically acceptable diluent, preservative, solubilizes, emulsifier, adjuvant, carrier and/or excipient.

For medical uses in the treatment of a patient in need thereof, an effective amount of the modified siRNA according to the invention, be it pure, encapsulated or protected or in a pharmaceutical composition, is to be used to treat or prevent any suitable pathological symptoms of diseases or disorders selected from the group comprising hepatitis, gastroenteritis, keratoconjunctivitis, cystitis, rhinitis, pharyngitis, diarrhea, and respiratory diseases.

According to the invention the term “treat or prevent”, or any lingual variation thereof, as used herein refers to preventing the manifestation of symptoms before they occur, slowing down the progression of the disease, slowing down the deterioration of symptoms, enhancing the onset of remission period, slowing down the irreversible damage caused in the progressive chronic stage of the disease, delaying the onset of said progressive stage, reducing the severity or curing the disease, improving the survival rate or a more rapid recovery, preventing the disease form occurring or a combination of two or more of the above.

According to the invention, an “effective amount” of the modified siRNA is an amount for achieving treatment or prevention of any of beforementioned diseases or disorders.

The present invention may be administered by various routes. Examples of such routes, without limitation may be intravenous, subcutaneous, intraocularly and/or topical.

In summary the inventors could show that the modified siRNA following the disclosed modification pattern and protected by e.g. LNP has a significantly increased bioavailability and higher stability against degradation enables in vivo and reduces effectively viral replication based on post transcriptional gene silencing via RNA interference (RNAi). The siRNA sequences selected show high efficiency and can be used against a wide variety of hAd subclasses.

### DESCRIPTION OF THE FIGURES

FIG. 1|Evaluation of anti-adenoviral siRNAs sipTP, siPol-1 and siPol-2.

(A) Schematic representation of the tested anti-adenoviral siRNAs showing both the sense and antisense strands of sipTP, siPol-1, siPol-2, and the non-silencing control siRNA siContr.

(B) Determination of inhibition of hAd5 replication with anti-adenoviral siRNAs by quantitative real-time PCR. Hela cells were infected with hAd5 at a MOI of 0.1, 1 and 2.5 and after 2 h transfected with 30 nM sipTP, siPol-1, siPol-2, siContr or with transfection reagent only (w/o siRNA). After 48 h cells were lysed. Supernatants were used for infection of Hela cells that were lysed after 2 h and their supernatants used for quantification of infectious adenoviral genomes by quantitative real-time PCR. Fold-change was calculated using the ΔΔCt method against siContr-treated cells with determination of genomic DNA of 18S rRNA for normalization. Significance against siContr-treated cells: ***p<0.001.

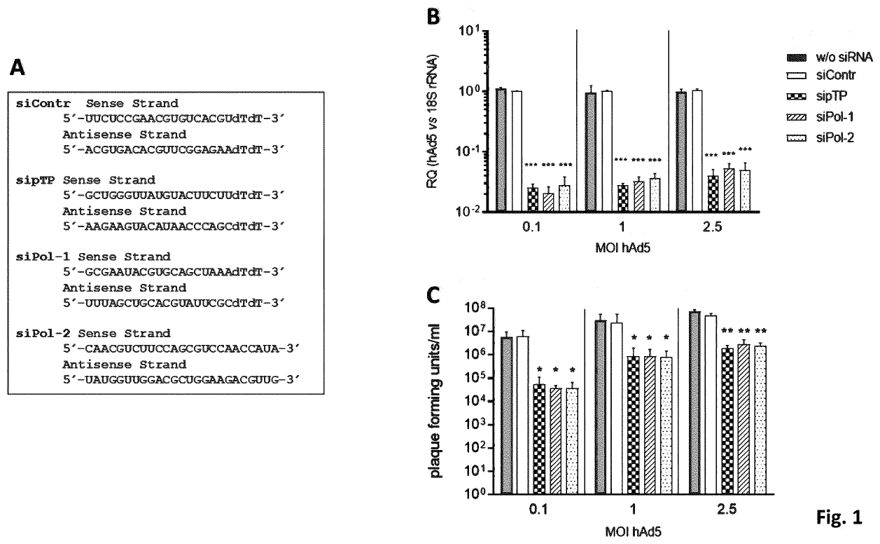

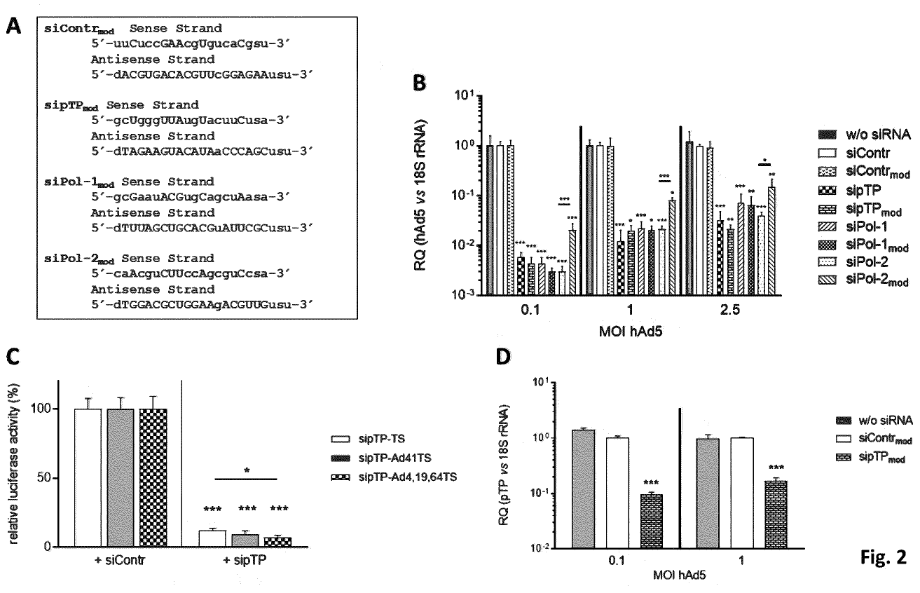

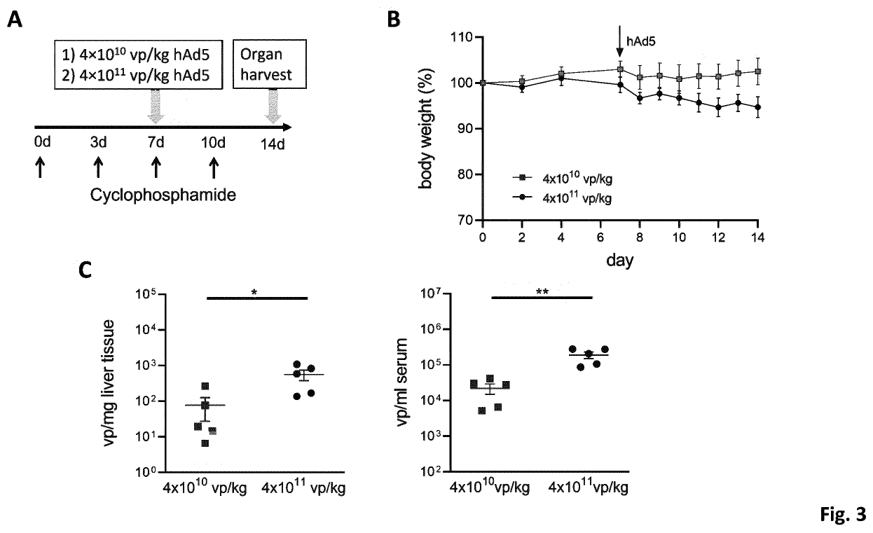

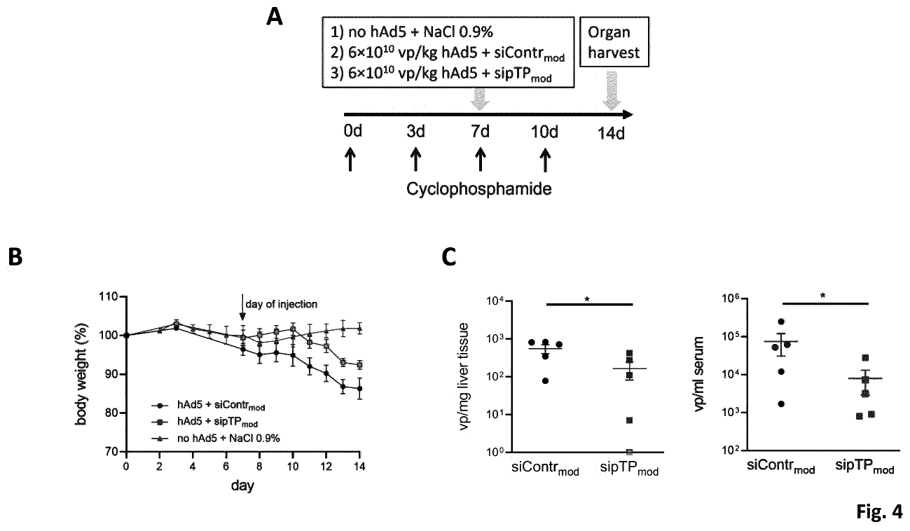

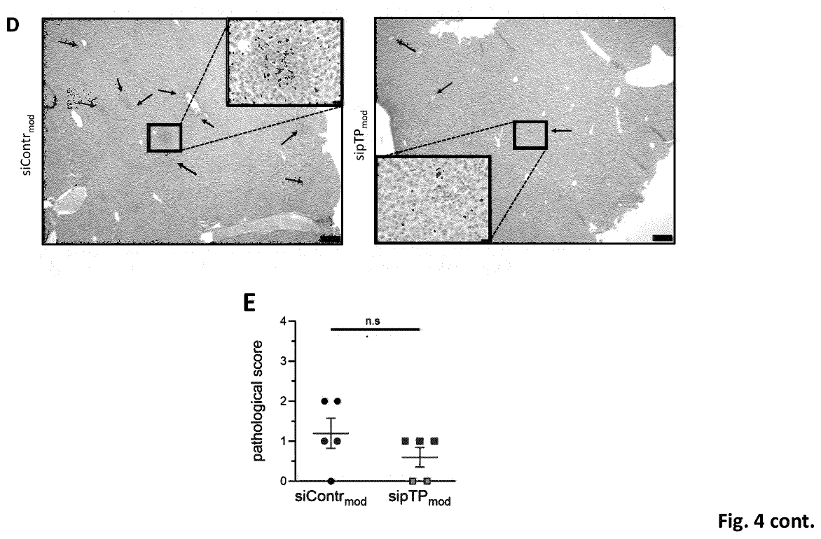

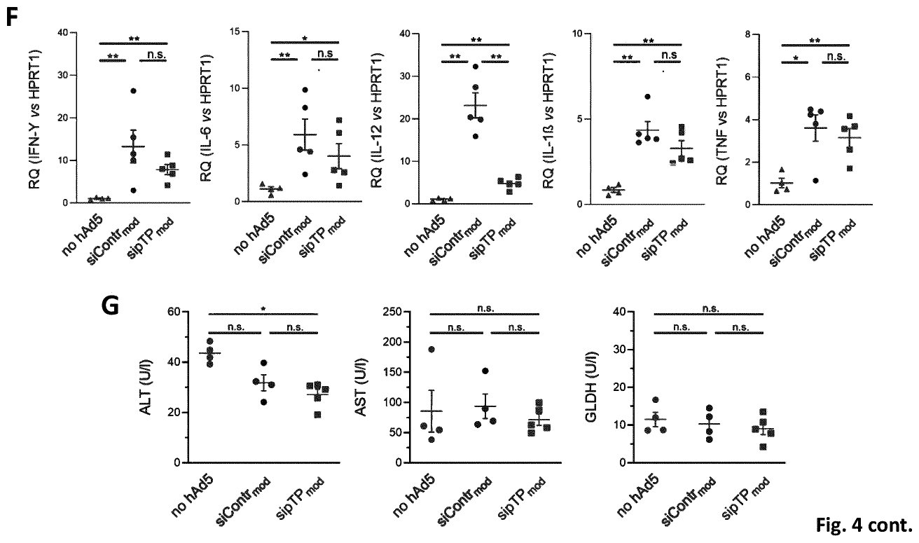

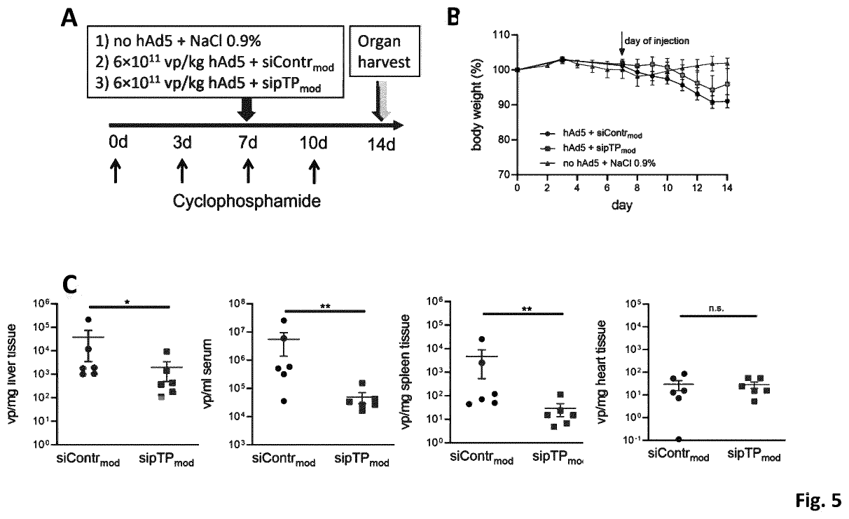

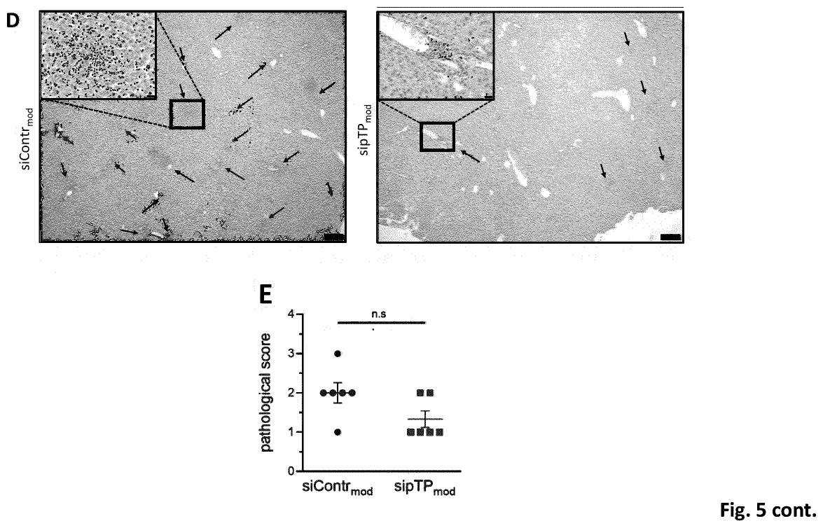

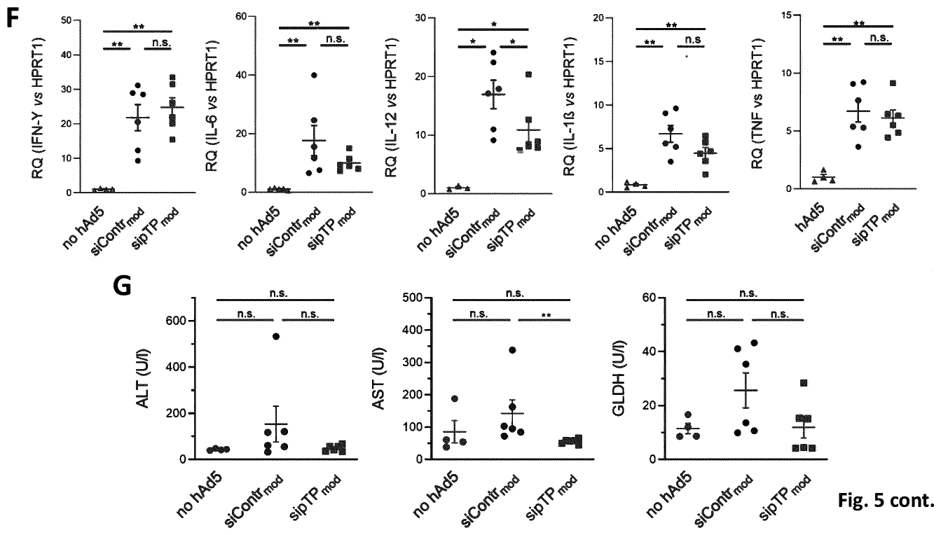

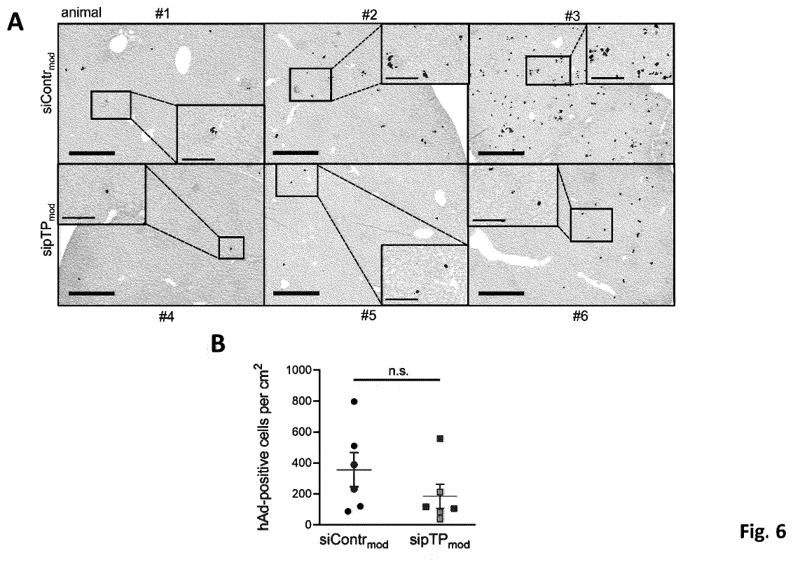

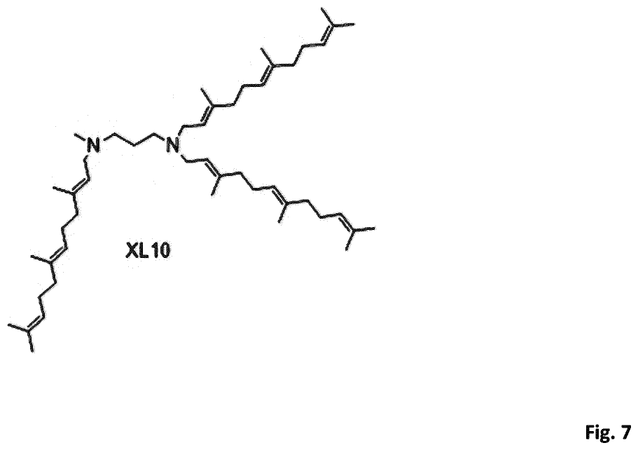

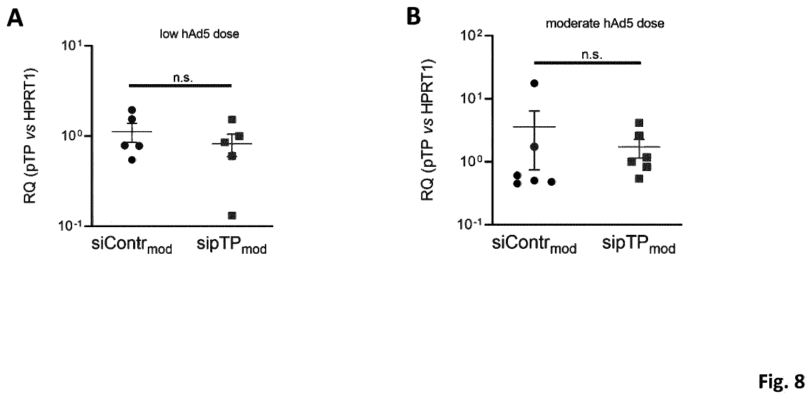

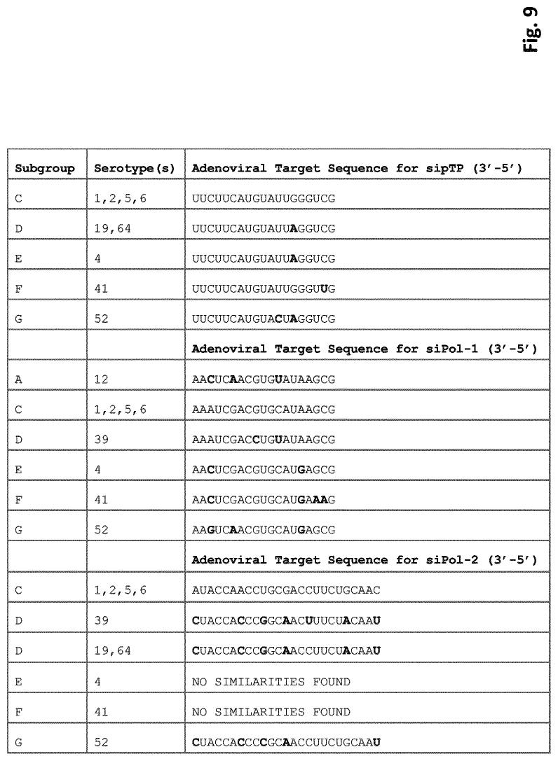

(C) Determination of inhibition of hAd5 replication with anti-adenoviral siRNAs by plaque assay. Hela cells were treated as described under FIG. 1B. Supernatants containing infectious hAd5 were directly quantified by plaque assay. Significance against siContr-treated cells: *p<0.05 and **p<0.01.

FIG. 2|Evaluation of anti-adenoviral siRNAs sipTP_{mod}, siPol-1_{mod}, siPol-2_{mod} and silencing of pTP of different adenoviral serotypes by sipTP.

(A) Schematic representation of tested modified anti-adenoviral siRNAs sipTP_{mod}, siPol-1_{mod}, siPol-2_{mod} and control siRNA (siContr_{mod}). The sense and antisense strands of the sequences are shown; N: RNA residues, dN: DNA residues, n: 2′-O-methyl residues, s: phosphorothioate.

(B) Inhibition of hAd5 replication by modified anti-adenoviral siRNAs. siRNAs described under FIG. 1B and their modified versions sipTP_{mod}, siPol-1_{mod}, siPol-2_{mod} and siContr_{mod} were used to infect Hela cells and to quantify its inhibitory effect on hAd5 replication as described under FIG. 1B, Significance of unmodified siRNAs versus siContr and modified siRNAs versus siContr_{mod} or as indicated: ***p<0.001, **p<0.01, *p<0.05.

(C) Silencing of pTP of different hAd serotypes by sipTP in a reporter gene assay. Hela cells were co-transfected with sipTP and a hRLuc reporter plasmid containing the complete corresponding sipTP target site (sipTP-TS) from hAd5, the target sites for the serotype hAd41 (sipTP-Ad41TS) or for hAd4, hAd19 and hAd64 (sipTP-Ad4,19,64TS). The cells were harvested 48 h after transfection and luciferase activity was determined. As a control, each sipTP-TS reporter plasmid was co-transfected with siContr. The siRNA silencing activity was calculated as a percentage of luciferase activity in samples treated with sipTP compared to samples treated with siContr. Significance against siContr-treated cells or as indicated: ***p<0.001, *p<0.05.

(D) Determination of adenoviral pTP mRNA expression after treatment with anti-adenoviral siRNAs by quantitative real-time RT-PCR. Hela cells were infected with hAd5 at a MOI of 0.1 and 1 and after 2 h transfected with 30 nM sipTP_{mod}, siContr_{mod} or transfection reagent alone (w/o siRNA). After 48 h total RNA was isolated and levels of pTP mRNA quantified. Fold-change was calculated using the ΔΔCt method against siContr_{mod}-treated cells with determination of cellular 18S rRNA levels for normalization. Significance against siContr_{mod}-treated cells: ***p<0.001.

FIG. 3|Dose-dependent replication of hAd5 in immunosuppressed Syrian hamster.

(A) Time course of cyclophosphamide (CP) and hAd5 injection. Syrian hamsters were divided into two groups (each n=5 animals) and immunosuppressed by repeated application of CP, as indicated. Both groups were infected with hAd5 at day 7 after first CP injection via i.v. injection of the virus into the jugular vein. One group received hAd5 at a dose of 4×10¹⁰ vp/kg and the other group hAd5 at a dose of 4×10¹¹ vp/kg. Animals were sacrificed and analyzed on day 14 after first CP injection.

(B) Development of body weight of immunosuppressed, hAd5-infected Syrian hamsters. Shown are the changes in body weight, beginning after the first CP injection. The time of adenoviral infection is marked by an arrow.

(C) Measurement of liver tissue and serum titers of hAd5. Titers of infectious hAd5 in liver tissue and serum were determined by an in vitro hAd5 amplification/quantitative real-time PCR protocol, as described in the Examples section. Significance: *p<0.05, **p<0.01.

FIG. 4|Application of LNP-sipTP_{mod} concomitant with hAd5 to immunosuppressed Syrian hamster decreases hAd5 infection of the liver in a low dose hAd5 infection model.

(A) Time course of CP application, hAd5 and LNP-siRNA injection. Syrian hamsters were divided into three groups and immunosuppressed by repetitive CP application as indicated. One group received no hAd5 but 0.9% NaCl (n=4) and two groups were infected with hAd5 at a dose of 6×10¹⁰ vp/kg at day 7 after first CP injection by injection of the virus into the left jugularis vein. Simultaneously, animals in one of the hAd5-infected groups received 2 mg/kg LNP-siContr_{mod} (siContr_{mod}) (n=5), whereas the other group received 2 mg/kg LNP-sipTP_{mod} (sipTP_{mod}) (n=5). In each case, application was into the right jugular vein. All animals were sacrificed and analyzed at day 14 after beginning of CP administration.

(B) Development of body weight of immunosuppressed Syrian hamsters. Shown are relative changes of body weight beginning at first CP injection. Time of virus/siRNA injection is marked by an arrow.

(C) The titers of hAd5 measured in liver tissue and serum. Titers of hAd5 in liver tissue and serum were determined by an in vitro hAd5 amplification/quantitative real-time PCR protocol, as described in the Examples section. Significance: *p<0.05.

(D) Severity of liver tissue damage. H&E staining of formalin-fixed liver slides. Shown is a representative of each hAd5-infected group. Arrows show foci of inflammation. Scale bars, 200 μm; scale bars of magnification, 20 μm.

(E) Liver damage was assessed and given as a pathological score presenting a scale from 0 (no damage) to 5 (severe damage). n.s., not significant.

(F) Expression of inflammatory and immune mediators in liver. Samples were assessed for type II interferon response-related (IFN-γ) and innate/adaptive mediator (IL-6, IL-12, IL-13, TNF, IFN-γ) expression by quantitative real-time RT-PCR. Fold-change was calculated via the ΔΔCt method against uninfected, immunosuppressed animals with the housekeeping gene HPRT1 used to normalize the sample. Significance, *p<0.05; **p<0.01; n.s., not significant.

(G) ALT, AST and GLDH levels in sera of the three investigated groups. Significance, *p<0.05; n.s., not significant. Note: serum of only 4 animals in the LNP-siContr_{mod}-treated group could be analyzed.

FIG. 5|Application of LNP-sipTP mod concomitant with hAd5 to immunosuppressed Syrian hamster decreases hAd5 infection of the liver in a moderate dose hAd5 infection model.

(A) Syrian hamsters were divided into three groups and treated and analyzed as shown in FIG. 4A with following exceptions: hAd5 was administered at a dose of 6×10¹¹ vp/kg and each group of hAd5-infected animals comprised 6 animals.

(B) Development of body weight of immunosuppressed Syrian hamsters. Shown are relative changes of body weight beginning at first CP injection. Time of virus/siRNA injection is marked by an arrow.

(C) The titers of hAd5 measured in liver tissue, serum, spleen, and heart tissue are shown. Determination of hAd5 titers and testing of significances were performed as described in FIG. 4C. Significance: *p<0.05 and **p<0.01; n.s., not significant.

(D) The severity of liver tissue damage is shown in H&E-stained formalin-fixed liver slides. A representative example of each hAd5-infected group is shown. The foci of inflammation are indicated by arrows. Scale bars, 200 μm; scale bars of magnification, 20 μm.

(E) The liver damage shown here was assessed and given as a pathological score, as described under FIG. 4E.

(F) The expression of IL-6, IL-12, IL-1B, TNF and IFN-g in the liver was determined as described under FIG. 4F. Significance: *p<0.05, **p<0.01; n.s., not significant.

(G) Shown are the ALT, AST and GLDH levels measured in sera of the three investigated groups. Significance: **p<0.01; n.s., not significant.
Note: The data shown for the uninfected group 1 (no hAd5) are the same as those shown in FIGS. 4B, F and G.

FIG. 6|Detection of hAd5 in liver tissue by immunohistochemistry.

Liver tissues were stained for expression of hAd5 E1A protein using an E1A-specific antibody. Ad5 infected cells are characterized by a dark brown color.

(A) Shown are images of stained liver tissue of each three representatives per group. Scale bars, 600 μm; scale bars of magnification, 200 μm.

(B) Comparison of hAd5-positive cells in liver tissue of hAd5-infected and LNP-sipTP_{mod}- or LNP-siContr_{mod}-treated animals. The number of positive cells was set in relation to the area of the section. Significance: n.s., not significant.

FIG. 7|Example of a lipid nanoparticles (LNP).

Chemical structure of aminolipid XL-10.

FIG. 8|Determination of hAd5 PTP mRNA levels in liver tissue by quantitative real-time RT-PCR.

Total RNA of liver tissues of LNP-siContr-treated and LNP-sipTP-treated animals was isolated in mod mod the low hAd5 dose experiment (A) and for the moderate hAd5 dose experiment (B) and levels of pTP mRNA quantified by using pTP-specific primers. Fold change was calculated using the ΔΔCt method against LNP-siContr_{mod}-treated animals with determination of HPRT1 mRNA levels for normalization. Significance as indicated: n.s., not significant.

FIG. 9|Sequence comparison of ipTP, siPol-1 and siPol-2

The sequence comparison demonstrates that ipTP, siPol-1 and siPol-2 perfectly matched their respective target sequences in the pTP and Pol mRNA of the adenoviral subgroup C serotypes 1, 2, 5, and 6. In other subtypes one or several mismatches occur, At least one mismatch (if not up to 4 mismatches-data not shown) is tolerated by sipTP as shown in FIG. 2C. Thus, the siRNAs of the invention can successfully and safely target also other hAd than that of the hAd subgroup C.

TABLE 1
Concordance list regarding the sequences disclosed in the application and the numbering of the
sequence listing.

| | Sequence information | |
|---|---|---|
| | (Note: while the sequence listing does not | |
| | allow uracil in RNA sequences the | |
| | specification still discloses RNA | |
| SEQ | sequences with U = uracil, so does this | |
| ID | table) | Description |
| 1 | TCGAGGCTGGGTTATGTACTTCTTCTTT | Artifical sequence, oligonucleotid primer |
| 2 | AAAGAAGAAGTACATAACCCAGCC | Artifical sequence, oligonucleotid primer |
| 3 | TCGAGGTTGGGTTATGTACTTCTTCTTT | Artifical sequence, oligonucleotid primer |
| 4 | AAAGAAGAAGTACATAACCCAACC | Artifical sequence, oligonucleotid primer |
| 5 | TCGAGGCTGGATTATGTACTTCTTCTTT | Artifical sequence, oligonucleotid primer |
| 6 | AAAGAAGAAGTACATAATCCAGCC | Artifical sequence, oligonucleotid primer |
| 7 | CACATCCAGGTGCCTCAGAA | Artifical sequence, oligonucleotid primer |
| 8 | AGGTGGCGTAAAGGCAAATG | Artifical sequence, oligonucleotid primer |
| 9 | CCCCTCGATGCTCTTAGCTG | Artifical sequence, oligonucleotid primer |
| 10 | TCGTCTTCGAACCTCCGACT | Artifical sequence, oligonucleotid primer |
| 11 | TCAACTTCGCCGTGGACTTCT | Artifical sequence, oligonucleotid primer |
| 12 | AGGTAGTTGAGGGTGGTGGC | Artifical sequence, oligonucleotid primer |
| | The following sequences are disclosed in | |
| | FIG. 9.: (Note: in the experimental data the | |
| | sequences are presented in 3′→5′, here | |
| | also the corresponding 5′→3′ is listed) | |
| 13 | UUCUUCAUGUAUUGGGUCG | Adenoviral Target Sequence sipTP (3′-5′): |
| | 5→3: GCUGGGUUAUGUACUUCUU | Serotype(s) C 1, 2, 5, 6 |
| 14 | UUCUUCAUGUAUUAGGUCG | Adenoviral Target Sequence sipTP (3′-5′): |
| | 5→3: GCUGGAUUAUGUACUUCUU | Serotype(s) D 19, D 64 |
| 15 | UUCUUCAUGUAUUAGGUCG | Adenoviral Target Sequence sipTP (3′-5′): |
| | 5→3: GCUGGAUUAUGUACUUCUU | Serotype(s) E 4 |
| 16 | UUCUUCAUGUAUUGGGUUG | Adenoviral Target Sequence sipTP (3′-5′): |
| | 5→3: GUUGGGUUAUGUACUUCUU | Serotype(s) F 41 |
| 17 | UUCUUCAUGUACUAGGUCG | Adenoviral Target Sequence sipTP (3′-5′): |
| | 5→3: GCUGGAUCAUGUACUUCUU | Serotype(s) G 52 |
| 18 | AACUCAACGUGUAUAAGCG | Adenoviral Target Sequence for siPol-1 |
| | 5→3: GCGAAUAUGUGCAACUCAA | (3′-5′): Serotype(s) A 12 |
| 19 | AAAUCGACGUGCAUAAGCG | Adenoviral Target Sequence siPol-1 (3′-5′): |
| | 5→3: GCGAAUACGUGCAGCUAAA | Serotype(s) C 1,2,5,6 |
| 20 | AAAUCGACCUGUAUAAGCG | Adenoviral Target Sequence for siPol-1 |
| | 5→3: GCGAAUACGUGCAGCUAAA | (3′-5′): Serotype(s) D 39 |
| 21 | AACUCGACGUGCAUGAGCG | Adenoviral Target Sequence for siPol-1 |
| | 5→3: GCGAGUACGUGCAGCUCAA | (3′-5′): Serotype(s) E 4 |
| 22 | AACUCGACGUGCAUGAAAG | Adenoviral Target Sequence for siPol-1 |
| | 5→3: GAAAGUACGUGCAGCUCAA | (3′-5′): Serotype(s) F 41 |
| 23 | AAGUCAACGUGCAUGAGCG | Adenoviral Target Sequence for siPol-1 |
| | 5→3: GCGAGUACGUGCAACUGAA | (3′-5′): Serotype(s) G 52 |
| 24 | AUACCAACCUGCGACCUUCUGCAAC | Adenoviral Target Sequence for siPol-2 |
| | 5→3: CAACGUCUUCCAGCGUCCAACCAUA | (3′-5′): Serotype(s) C 1, 2, 5, 6 |
| 25 | CUACCACCCGGCAACUUUCUACAAU | Adenoviral Target Sequence for siPol-2 |
| | 5→3: UAACAUCUUUCAACGGCCCACCAUC | (3′-5′): Serotype(s) D 39 |
| 26 | CUACCACCCGGCAACCUUCUACAAU | Adenoviral Target Sequence for siPol-2 |
| | 5→3: UAACAUCUUCCAACGGCCCACCAUC | (3′-5′): Serotype(s) D 19, D 64 |
| 27 | CUACCACCCCGCAACCUUCUGCAAU | Adenoviral Target Sequence for siPol-2 |
| | 5→3: UAACGUCUUCCAACGCCCCACCAUC | (3′-5′): Serotype(s) G 52 |
| | The following sequences are disclosed in | |
| | FIG. 1.: d = DNA nucleotides | |
| 28 | UUCUCCGAACGUGUCACGUdTdT | siContr Sense Strand |
| 29 | ACGUGACACGUUCGGAGAAdTdT | siContr Antisense Strand |
| 30 | GCUGGGUUAUGUACUUCUUdTdT | sipTP Sense Strand |
| 31 | AAGAAGUACAUAACCCAGCdTdT | sipTP Antisense Strand |
| 32 | GCGAAUACGUGCAGCUAAAdTdT | siPol-1 Sense Strand |
| 33 | UUUAGCUGCACGUAUUCGCdTdT | siPol-1 Antisense Strand |
| 34 | CAACGUCUUCCAGCGUCCAACCAUA | siPol-2 Sense Strand |
| 35 | UAUGGUUGGACGCUGGAAGACGUUG | siPol-2 Antisense Strand |
| | The following sequences are disclosed in | |
| | FIG. 2.: capital letters = unmodified | |
| | nucleotides; lowercase = 2′-O-methyl | |
| | residues; s = phosphorothioate; d = DNA | |
| | nucleotides | |
| 36 | uuCuccGAAcgUgucaCgsu | siContrmod Sense Strand |
| 37 | dACGUGACACGUUcGGAGAAusu | siContrmod Antisense Strand |
| 38 | gcUgggUUAugUacuuCusa | sipTPmod Sense Strand |
| 39 | dTAGAAGUACAUAaCCCAGCusu | sipTPmod Antisense Strand |
| 40 | gcGaauACGugCagcuAasa | siPol-1mod Sense Strand |
| 41 | dTUUAGCUGCACGuAUUCGCusu | siPol-1mod Antisense Strand |
| 42 | caAcguCUUccAgcguCcsa | siPol-2mod Sense Strand |
| 43 | dTGGACGCUGGAAgACGUUGusu | siPol-2mod Antisense Strand |

### EXAMPLES

The following examples illustrate viable ways of carrying out the invention as intended, without the intent of limiting the invention to said examples.

### General Experimental and Analytical Techniques for the Subsequent Examples

Cell culture. HEK293 (human embryonic kidney) cells were cultured in high glucose Dulbecco's Modified Eagle Medium (DMEM, Biowest, Darmstadt, Germany) supplemented with 10% fetal calf serum (FCS; c.c. pro GmbH, Oberdorla, Germany), L-Glutamine (Sigma-Aldrich, Merck KGaA, Darmstadt, Germany), Sodium pyruvate (Sigma-Aldrich) and 1% each of penicillin and streptomycin (AppliChem GmbH, Darmstadt, Germany). HeLa (human cervical carcinoma) cells were grown in Minimum Essential Medium (MEM, Gibco, Thermo Fisher Scientific, Inc., Waltham, MA, USA) with L-Glutamine and supplemented with 5% FCS, 1% each of penicillin and streptomycin, 10 mM HEPES (Sigma-Aldrich) and 0.1 mM NEAA (Thermo Fisher Scientific).

Production of human adenovirus 5. HAd5 stock batch was a kind gift from Stefan Weger (Institute of Virology, Campus Benjamin Franklin, Charité-Universitätsmedizin Berlin, Berlin, Germany). HAd5 was amplified on HEK293 cells, concentrated and purified by CsCl gradient centrifugation and desalted with PD-10 desalting columns (Cytiva Life Sciences, Freiburg im Breisgau, Germany). The viral titers were determined by photometric measurement of the optical density at 260 nm to count virus particles (vp)/ml and by standard plaque assay to count plaque forming units (pfu)/ml on HEK293 cells.

siRNAs. The online tool BLOCK-iTT RNAi Designer from ThermoFisher Scientific was used to select new siRNA against adenoviral pTP and pol genes resulting in the design of the sipTP and siPol-1, respectively. The siPol-2, which is also directed against the pol gene and siContr which does not match any sequence present in the viral or human genome has been described previously. The siRNAs were synthesized as siRNA duplexes with dTdT 3′-overhangs (Eurofins Genomics Germany GmbH, Ebersberg, Germany). Chemically modified siRNAs sipTP_{mod}, siPol-1_{mod}, siPol-2_{mod}, siContr_{mod} Were incorporated by Axolabs GmbH (Kulmbach, Germany). The sequences of unmodified and chemically modified siRNAs are listed in FIGS. 1A and 2A.

Generation of LNPs with encapsulated siRNAs. For in vivo delivery, siContr_{mod} and sipTP_{mod} Were encapsulated within LNP, which contain the cationic aminolipid XL-10. The generation of the XL-10 containing LNP with encapsulated siRNAs has been described previously. Briefly, the T-junction-based produced LNPs were a composition of a lipid mixture containing the aminolipid XL-10,1,2-distearoyl-3-phosphatidylcholine (DSPC), α-[3′-(1,2-dimyristoyl-3-propanoxy)-carboxamide-propyl]-ω-methoxy-polyoxyethylene (PEG-c-DOMG), and cholesterol. The ratio of XL10:DSPC:Cholesterol:PEG-DOMG was 50:10:38.5:1.5 molar percent. The lipids were first mixed in ethanol and the siRNA molecules were dissolved in an aqueous buffer. The total lipid to siRNA ratio was 7:1. Then both mixtures were mixed together, which led to the self-assembly of the particles encapsulating the siRNAs. The particle size, the polydispersity index and the Zeta potential were determined by dynamic light scattering (DLS) method. The drug concentration was determined by measurement the OD₂₆₀ and the drug encapsulation by the Oligogreen assay. Generation of LNP-siRNAs and all measurements on the LNP-siRNAs were carried out by Axolabs GmbH, Kulmbach, Germany.

Plasmids. Plasmids containing miR-TS were generated by insertion of annealed miR-TS primers into the 3′-UTR of Renilla luciferase (hRLuc) reporter cDNA psiCheck2 (Promega GmbH, Walldorf, Germany) via Xhol and Pmel restriction sites.

The primers were for sipTP-TS,
(SEQ ID: No. 1)
5′-TCGAGGCTGGGTTATGTACTTCTTCTTT-3′
and
(SEQ ID: No. 2)
5′-AAAGAAGAAGTACATAACCCAGCC-3′,
for sipTP-Ad41TS
(SEQ ID: No. 3)
5′-TCGAGGTTGGGTTATGTACTTCTTCTTT-3′
and
(SEQ ID: No. 4)
5′-AAAGAAGAAGTACATAACCCAACC-3′,
for sipTP-Ad4,19,64TS
(SEQ ID: No. 5)
5′-TCGAGGCTGGATTATGTACTTCTTCTTT-3′
and
(SEQ ID: No. 6)
5′-AAAGAAGAAGTACATAATCCAGCC-3′.
The correctness of all plasmids was confirmed by
sequencing.

Luciferase reporter assays for detection of siRNA activity. HEK293 cells were seeded in 48-well plates. The next day cells of one well were transfected with 50 ng dual luciferase reporter plasmids containing the corresponding miR-TS and 30 nM of siRNAs using Lipofectamine 2000 transfection reagent (Thermo Fisher Scientific). Firefly luciferase and hRLuc activity were analyzed after 48 h using Dual Luciferase Reporter System (Promega GmbH) in a Lumat LB 9507 Luminometer (Berthold Technologies Gmbh & Co. KG, Bad Wildbad, Germany), as recommended by the manufacturer.

Transfection of siRNAs and hAd5 infection. Hela cells were seeded in 24-well-plates. The next day cells were infected with selected MOIs of hAd5 for 2 h in serum-free medium. Medium was replaced by complete medium and siRNAs were transfected to a final concentration of 30 nM per well via Lipofectamine RNAiMAX transfection reagent (Thermo Fisher Scientific). After 48 h cells were lysed by three freeze-thaw cycles. Supernatants were used to directly quantify infectious hAd5 via plaque assay or to infect new Hela cells seeded in 24-well-plates for 2 h to determine the amount of infectious hAd5 particles by subsequent quantitative real-time PCR.

Plaque assay. HEK293 cells were seeded in 12-well-plates and reached a confluent monolayer the next day. Cells were inoculated with log dilutions of virus containing solution in serum-free medium for 1 h. Supernatant was discarded, and cells were overlaid with a 1:3 mixture of 5% low melting agarose (Sigma-Aldrich) and complete medium. After 10 to 14 days plaques appeared and hAd5 titer was determined as pfu per ml.

Quantitative real-time PCR for detection of adenoviral DNA. After discarding the supernatant of hAd5 infected cells, the cells were lysed in PBS by three freeze thaw cycles and transferred to a fresh tube, heat-inactivated at 95 °C for 10 min and centrifugated at 12,000×rpm for 10 min. 1.5 μl of the supernatant were used directly in a quantitative real-time PCR for detection of hAd5 DNA using primers 5′-CACATCCAGGTGCCTCAGAA-3′ (SEQ ID: No. 7) and 5′-AGGTGGCGTAAAGGCAAATG-3′ (SEQ ID: No. 8) directed against adenoviral hexon gene and the SsoFast™ EvaGreen® Supermix (Bio-Rad Laboratories GmbH, Feldkirchen, Germany). As reference the genomic DNA of 18S rRNA was analyzed using the primer pair 5′-CCCCTCGATGCTCTTAGCTG-3′ (SEQ ID: No. 9) and 5′-TCGTCTTCGAACCTCCGACT-3′ (SEQ ID: No. 10). Quantitative real-time PCR reaction was carried out in duplicate in a C1000™ Thermal Cycler and CFX96™ Real-Time-System (Bio-Rad Laboratories GmbH). Relative hAd5 genome copy numbers were determined by the ΔΔCt calculation method.

Determination of infectious hAd5 titers in tissue. Three pieces of each animal organ (liver, spleen) and one piece of heart were separately homogenized in 0.4 ml DMEM using disposable plastic pestles followed by two freeze-thaw cycles. Serum from animals was obtained by centrifugation of whole blood and stored at −20 °C. HAd5 titer was determined as described previously. Briefly, Hela cells were seeded in 24-well-plates and the next day incubated with 1:10 diluted virus solution for 2 h in serum-free medium. The medium was replaced by complete medium and after 48 h cells were washed with PBS and virus was released from cells by three freeze-thaw cycles in PBS. For absolute quantification of viral titers, Hela cells were in parallel infected with 500, 50, 5 and 0.5 vp hAd5 per cell (hAd5 standard). The number of viral genomes was determined by quantitative real-time PCR as described above.

Quantification of hAd5 PTP mRNA and immune mediator mRNA. Total RNA was isolated from liver tissue using TRIzol (Thermo Fisher Scientific) according to the manufacturer's recommendations. Two mg of the RNA were treated with 2 to 3 U DNasel (New England Biolabs GmbH, Frankfurt am Main, Germany) for 1 to 2 h. High-Capacity cDNA Reverse Transcription Kit (Thermo Fisher Scientific) was used to reverse transcribe 400 to 800 ng DNasel-treated RNA. hAd5 pTP mRNA was determined by quantitative real-time RT-PCR using the primer pair 5′-TCAACTTCGCCGTGGACTTCT-3′ (SEQ ID: No. 11) and 5′-AGGTAGTTGAGGGTGGTGGC-3′ (SEQ ID: No. 12) with 18S rRNA expression for normalization of the in vitro data or with HPRT1 expression for normalization of the in vivo data. IL-6, IL-12, IL-1B, TNF and IFN-γ expression were determined by quantitative real-time RT-PCR using previously described primer pairs recognizing the respective sequences with determination of HPRT1 for normalization. The SsoFast™ EvaGreen® Supermix was used and change of expression was calculated using the ΔΔCt method against siContr_{mod}-transfected Hela cells for evaluation of the in vitro data or against NaCl 0.9%-treated and not hAd5-infected immunosuppressed animals.

Measurement of ALT, AST and GLDH. Serum analysis for ALT, AST and GLDH activity was done by Laboklin GmbH & Co. KG, Bad Kissingen, Germany. All in vivo procedures involving the use and care of animals were performed according to the European principles of laboratory animal care (Directive 2010/63/EU) and approved by the local ethics committee (Landesamt für Gesundheit und Soziales, Berlin, Germany).

Animal experiments. Male Syrian hamsters were obtained from Charles River Laboratories (Sulzfeld, Germany) and had a weight of about 110 g when starting immunosuppression with cyclophosphamide (CP, Sigma-Aldrich). CP was administered i.p. at an initial dose of 140 mg/kg, and then twice weekly at a dose of 100 mg/kg. Seven days after first CP injection, animals were anesthetized with isoflurane, the left jugular vein was prepared and hAd5 was injected at a dose of 4×10¹⁰ vp/kg or 4×10¹¹ vp/kg hAd5. In another experiment 6×10¹⁰ vp/kg of hAd5 (low dose infection model) or 6×10¹¹ vp/kg of hAd5 (moderate dose infection model) were i.v. injected 7 days after first CP injection. In this experiment, the right jugular vein was prepared immediately after hAd5 infection and 2 mg/kg of the LNP-siRNA were injected. Immunosuppressed animals that were not hAd5 infected, received NaCl 0.9% into the left jugularis vein 7 days after first CP injection. The surgical step was accompanied by analgesic treatment with subcutaneous injection of 0.5 mg/kg Meloxicam (Mesolute 5 mg/ml, CP-Pharma Handelsgesellschaft mbH, Burgdorf, Germany). Animals were sacrificed for organ harvest 14 days after first CP injection. The organs were dissected and rapidly frozen in liquid nitrogen or placed in 4% formalin.

Immunohistological examination. Formalin-fixed tissues were paraffin embedded (FFPE), cut and stained with hematoxylin and eosin (HE) as previously described. A semiquantitative histopathologic scoring was performed by a board-certified pathologist to quantify inflammatory lesions. A grade of 0 was given to a completely physiologic liver tissue. Grade 1 showed minimal (1-2 spots), grade 2 mild (2-5 spots), grade 3 moderate (5-10 spots), grade 4 severe (>10 spots) and grade 5 severe (<10 spots and/or large coalescing areas) of inflammation. Preparation of 4- to 5-μm sections of FFPE livers for immunohistochemical staining was performed as previously described. Briefly, after blocking nonspecific antibody binding, sections were incubated overnight at 4 °C with a monoclonal mouse-anti adenovirus antibody (ab3648, Abcam, Cambridge, UK) that recognizes the hAd5 E1A protein. Afterwards slides were incubated at room temperature for 30 min with a biotinylated secondary goat-anti-mouse antibody (dilution 1:200 each; Vector Laboratories, Burlingame, CA, USA). An avidin-biotin-immunoperoxidase system (Vectastain Elite ABC Kit; Vector Laboratories, Eching, Germany) was used for immunolabeling and Diaminobenzidine tetrahydrochloride (DAB; Merck, Darmstadt, Germany) was used for viral protein visualization.

Statistics. Results are expressed as means±SEM. To test for statistical significance of in vitro data, an unpaired Student's t-test was applied. Statistical significance of in vivo data was determined using the Mann-Whitney U test.

### Example 1: Evaluation of Anti-Adenoviral siRNA

Studies have defined the adenoviral pTP and Pol genes as the best target genes for RNAi therapeutics for the inhibition of hAd infection. Two siRNAs (siPol-1 and siPol-2) targeting the adenoviral Pol gene and one siRNA targeting the adenoviral pTP gene (sipTP) were designed (FIG. 1A). Both Pol and pTP gene target sequences were from the adenovirus serotype 5, as it belongs to the adenoviral subgroup C, the subgroup most frequently detected in patients with severe adenovirus infections. To compare their silencing efficiencies, Hela cells were infected with hAd5 at a MOI of 0.1, 1 or 2.5 and transfected with 30 nM of sipTP, siPol-1 or siPol-2. Quantitative real-time PCR to determine the number of viral genomes and plaque assays to determine the amount of infectious hAd5, showed that all three siRNAs had a strong effect in inhibiting adenoviral infection in vitro (FIG. 1B, C). Relative to cells transfected with a non-silencing control siRNA (siContr), the adenoviral replication was inhibited by the siPol-1, siPol-2 and sipTP by approximately 98% (MOI 0.1) to 95% (MOI 2.5), as determined by quantitative real-time PCR, and by approximately 2 (MOI 0.1) to 1.5 (MOI 2.5) orders of magnitude, as determined by plaque assay. Thus, all the siRNAs show high efficiency against hAd5.

To achieve a high inhibitory efficiency in vivo the siRNAs siPol-1_{mod}, SiPol-2_{mod} and sipTP_{mod} Were designed (FIG. 2A). Selected positions of these siRNAs were modified with 2′-O-methyl to suppress an immune response against the siRNA and to confer stabilization against endonucleolytic degradation. Furthermore, two phosphorothioate linkages were introduced at the two ends of each strand to further increase protection against exonucleolytic degradation. In addition, a single overhang structure at the 3′-end of the guide strand and the optimization of the thermodynamic profile by introducing a DNA T at position one of the guide strand serve to increase siRNA activity. HeLa cells were then transfected with 30 nM of siPol-1, siPol-2 and sipTP and their modified counterparts siPol-1_{mod}, siPol-2_{mod} and sipTP_{mod}, as well as with siContr and its modified counterpart (siContr_{mod}), and infected with 0.1, 1 or 2.5 MOI of hAd5 to clarify whether the modifications in the siRNAs affect their ability to inhibit hAd5 replication. Quantitative real-time PCR revealed similar inhibition of hAd5 replication induced by sipTP and sipTP_{mod} or siPol-1 and siPol-1_{mod}, respectively, indicating that modification of sipTP and siPol-1 had no effect on inhibition of hAd5 replication. In contrast, treatment of hAd5-infected cells with siPol-2_{mod} resulted in a 7-fold lower inhibition of hAd5 replication compared to the use of siPol-2. The lower efficiency of siPol-2_{mod} appears to be due to a shortening of siRNA from 25mer to 19mer rather than the modifications per se, as the modification pattern of siPol-2_{mod} Was identical to that of sipTP_{mod} and sPol-1_{mod} (FIG. 2B). Due to the lower efficiency, siPol-2_{mod} was not investigated further.

Since severe adenoviral infections in humans can be caused by different hAd serotypes, the use of a specific siRNA that effectively recognizes a target sequence in multiple hAd subtypes would be beneficial for clinical application. As shown in Table 1, sipTP and siPol-1 matched perfectly to their respective target sequences in the pTP and Pol mRNA of the adenoviral subgroup C serotypes 1, 2, 5 and 6. One mismatch within a target sequence is often tolerated by an siRNA, therefore the sipTP was examined, which had only one mismatch relative each to the pTP target sequences of hAd19 and hAd64 (subgroup D), hAd4 (subgroup E), and hAd41 (subgroup F), but not siPol-1, which has two or more mismatches to the Pol target sequence in these virus strains. Hela cells were co-transfected with sipTP and a luciferase reporter plasmid containing the corresponding pTP target sequences of the four hAd or, as control, with a luciferase reporter plasmid containing the pTP target sequence of hAd5. The pTP target sequences of hAd4, hAd19, hAd41 and hAd64 were efficiently recognized, as indicated by strong silencing of luciferase reporter. Interestingly, a common mismatch in the sipTP target sequence of hAd4, hAd19 and hAd64 seems to even have increased the silencing activity of sipTP (FIG. 2C). Finally, the RNA interference mechanism of sipTP_{mod} was verified by determining pTP mRNA expression in Hela cells infected with 0.1 and 1 MOI hAd5 and transfected with 30 nM sipTP_{mod} or siContr_{mod}. Compared to the control, treatment with sipTP_{mod} resulted in 90.5% and 83% lower expression of pTP mRNA, respectively, as detected by real-time RT-PCR, demonstrating a strong silencing effect of sipTP_{mod} (FIG. 2C).

Based on these results, it was shown that the sipTP has a strong and broad activity among several subclasses of the human adenovirus. Chemical modifications introduced into the siRNA sequence to increase the stability and bioavailability for use in a later therapeutic application in vivo did so far not decrease adenoviral replication in vitro. Thus, the in vivo results as presented in Example 4 and 5 were even more surprising.

### Example 2: Generation of a System for siRNA Delivery In Vivo

The delivery of siRNAs in vivo is a key challenge for the development of efficient siRNA therapies. LNPs, which consist of different lipid components and form <100 nm large particles, play an important role in this. While the surface of these particles is surrounded by PEG lipids and is weakly positively charged, there is a largely hydrophobic core of inverted lipid micelles inside, which contains the siRNA. It has been shown that LNPs containing the cationic aminolipid XL-10 (FIG. 7) are capable of specifically transporting siRNAs into hepatocytes after i.v. administration. Therefore, XL-10 was used in a cocktail with other lipids and sipTP_{mod} and siContr_{mod} to prepare the therapeutic LNP-sipTP_{mod} and the control LNP-siContr_{mod}, respectively. The total lipid to siRNA ratio was 7:1 in the generated particles. Several parameters of the LNP siRNA preparations were determined before their use, as they are crucial for their in vivo functionality. LNP-sipTP_{mod} and LNP-siContr_{mod} had a size of 83.2 and 88.4 nm, respectively, and the polydispersity index was 0.04 for each. The zeta potential was 0.7 mV for LNP-sipTP_{mod} and 1.3 mV for LNP-siContr_{mod}. The drug encapsulation reached 90% and 89% for LNP-sipTP_{mod} and LNP-siContr_{mod}, respectively. The drug concentration of both LNP-siRNAs was 1.0 mg/ml. Both LNP-siRNA preparations thus had very similar parameters and parameters in the range of typical LNP-siRNA preparations.

It has been shown that a lipid cocktail with defined parameters containing the cationic aminolipid XL-10 is capable of delivering the modified siRNA into hepatocytes. This is one additional but essential parameter for an efficient use of sipTP_{mod} as therapeutic agent in vivo.

### Example 3: Course of hAd5 Infection in Immunosuppressed Syrian Hamsters

The Syrian hamster model has been developed as a standard model for investigation of hepatic hAd infections and analysis of therapeutic efficiency of anti-adenoviral drugs and biologicals in vivo. This model was therefore chosen to determine the anti-adenoviral efficacy of LNP-sipTP_{mod}. In an initial experiment, the inventor investigated the course and severity of hAd infection in Syrian hamsters as a function of viral dose, since viral dose is a crucial factor affecting both disease parameters in the model. For this propose, Syrian hamsters were immunosuppressed with CP-twice weekly. Animals were infected intravenously (i.v.) with 4×10¹⁰ (low dose) or 4×10¹¹ (moderate dose) virus particles (vp) of hAd5 per kg. The animals were investigated for another 7 days (FIG. 3A). No deaths were observed during the study period. After hAd5 infection, body weight remained unchanged in the low dose group, whereas it decreased slightly in the animals that received the moderate hAd5 dose (FIG. 3B). Replicating hAd5 was detected in the liver and serum at both the low and moderate doses. Corresponding to the different doses of virus administered, the hAd5 titers in liver and serum were about one order of magnitude higher in the moderate dose group than in the low dose group (FIG. 3C).

These results demonstrate that systemic application of hAd5 at low and moderate doses induces productive hepatic hAd5 infection and the amounts of virus detected in the liver and serum correlate with the initial dose of virus injected. Without treatment, the human adenovirus can replicate in immunosuppressed Syrian hamster, causing weight loss at higher virus concentrations.

### Example 4: LNP-sipTP_{mod} Inhibits Replication in Low Dose hAd5 Infection Model In Vivo

For the investigation of the therapeutic efficacy of LNP-sipTP_{mod} in the low dose infection model, Syrian hamsters were divided into three groups and immunosuppressed a week prior to hAd5 infection. Two of the three groups were i.v. infected with 6×10¹⁰ vp/kg hAd5. One of these groups was concurrently i.v. injected with 2 mg/kg LNP-sipTP_{mod}, whereas the other received 2 mg/kg of LNP-siContr_{mod}. All animals were sacrificed 7 days later (FIG. 4A). During the investigational period of 7 days, both groups lost weight after infection, but weight loss was lower in the LNP-sipTP_{mod} group (up to 7.6%) compared to the LNP-siContr_{mod} group (up to 13.7%) (FIG. 4B). Determination of hAd5 titers in the liver and serum revealed a significant reduction of virus burden in animals that were treated with LNP-sipTP_{mod} compared to LNP-siContr_{mod}-treated animals (FIG. 4C). Indeed, hAd5 titers were 3.6-fold lower in the liver and 10-fold lower in serum of LNP-sipTP_{mod}-treated animals than in LNP-siContr_{mod}-treated animals. The latter indicates that LNP-sipTP_{mod} treatment reduced viremia. No changes were found for pTP-mRNA levels in the liver as detected by real-time RT-PCR (FIG. 8A). Spleen and heart were also investigated for hAd5 infections, but viral titers were too low to obtain evaluable data (results not shown). Histological examination of liver sections showed a reduced inflammation foci frequency with reduced foci size in LNP-sipTP_{mod}-treated animals compared to LNP-siContr_{mod}-injected animals (FIG. 4D). Accordingly, the pathological score of liver tissue tended to be lower in LNP-sipTP_{mod}-treated animals than in LNP-siContr_{mod}-treated animals (FIG. 4E). To characterize the inflammatory processes in the liver more comprehensively, a panel of inflammatory and immune mediators in the liver tissue was measured by quantitative real-time RT-PCR. Samples were assessed for type II interferon response-related mediator IFN-γ and the innate/adaptive mediators IL-6, IL-12, IL-1B and TNF. All mediators were upregulated in hAd5-infected animals compared to uninfected immunosuppressed Syrian hamsters. More importantly the expression of IL-12 was significantly reduced and expression of IFN-Y, IL-6, IL-1B tended to be lower in the LNP-sipTP_{mod}-treated compared to LNP-siContr_{mod}-treated animals (FIG. 4F). To further investigate the influence of the siRNA therapy on liver damage, the activity of the alanine aminotransferase (ALT), the aspartate aminotransferase (AST) and the glutamate dehydrogenase (GLDH) representing biomarkers for liver injury were measured in the serum of hAd5-infected animals treated with LNP-sipTP_{mod}, LNP-siContr_{mod}, and in uninfected control animals. There were no differences in enzyme activity between animal groups (FIG. 4G), suggesting that the liver damage induced by the low dose of hAd5 was too small to significantly increase enzyme activity.

In conclusion, it is shown that the viral replication of hAd5 in immunosuppressed Syrian hamsters is successfully inhibited after administration of LNP-sipTP_{mod}. Substantially lower virus titers were detected in liver and serum and reduced weight loss was seen over the experimental period compared to the control group. It was also confirmed that inflammatory and immune mediators in the liver were lower in infected hamsters treated with LNP-sipTP_{mod}.

### Example 5: LNP-sipTP_{mod} Inhibits Replication in Moderate Dose hAd5 Infection Model In Vivo

Encouraged by the data obtained in the low dose hAd5 infection model and to analyze whether LNP-sipTP mod can also combat a more severe hepatic hAd5 infection, we next investigated the therapeutic efficacy of LNP-sipTP_{mod} after infection of immunosuppressed Syrian hamsters with a 10-fold higher hAd5 dose of 6×10¹¹ vp/kg (FIG. 5A). In this moderate dose model both LNP-sipTP_{mod}-treated and LNP-siContr_{mod}-treated hAd5-infected animals lost body weight during the investigation period, but the latter lost more (FIG. 5B). A significant reduction in viral titer in the liver and serum of LNP-sipTP_{mod}-treated compared to LNP-siContr_{mod}-treated hAd5-infected animals was observed, whereas hepatic pTP-mRNA levels remained unchanged (FIG. 8B). Compared to the low dose model, the inhibitory effect of LNP-sipTP_{mod} was even stronger, with a 20-fold reduction of virus titers in the liver and a 100-fold reduction of virus titers in the serum (FIG. 5C). In addition, a 100-fold reduction of hAd5 titer was found in spleen with LNP-sipTP_{mod} treatment, whereas there was no difference in the heart. Reduction of hepatic hAd5 titers by siRNA treatment was confirmed by immunohistochemical staining of hAd5-positive cells in liver sections showing that LNP-sipTP_{mod}-treated animals tended to have fewer hAd5-positive cells than siContr_{rmod}-treated animals (FIG. 6). Compared to LNP-siContr_{mod}-treated animals, there was a slightly reduced liver pathology for animals treated with LNP-sipTP_{mod}, as indicated by a reduced pathological score (FIG. 5D/E). As in the low-dose model, IFN-γ, IL-6, IL-12, IL-1B, and TNF were upregulated by hAd5 infection in the liver, whereas IL-12 levels were markedly reduced and IL-6 and IL-1b tended to be expressed at lower levels in the liver tissue of LNP-sipTP_{mod}-treated animals compared with LNP-siContr_{mod}-treated animals (FIG. 5F). In contrast to the low dose model the activities of ALT, AST and GLDH tended to be higher in the liver of immunosuppressed hAd5 infected Syrian hamsters treated with LNP-siContr_{mod} compared to uninfected animals, indicating a hAd5 dose of 6×10¹¹ vp/kg was sufficient to induce severe liver injury (FIG. 5G). Interestingly, hAd5-infected hamsters treated with LNP-sipTP mod retained the AST levels to those of uninfected hamsters.

The moderate dose hAd5 infection model showed compared to the low dose model, additional virus titers in spleen and indicates severe liver injuries in immunosuppressed Syrian hamsters. Administration of LNP-sipTP_{mod} effectively inhibits the virus replication in the moderate dose model and was able to keep AST activity, an important indicator for liver damage, at a normal level.

This study explicitly demonstrates the high efficacy of the invention to treat adenoviral infections in vivo.

1-15. (canceled)

16. A modified siRNA consisting of a guide and a passenger strand, wherein the guide strand is subdivided into a 5′-region, a complementary region to an adenoviral target sequence, and a 3′-region, wherein the guide strand is characterized in having the following modification pattern:
5′-X_{d}-Y-Y-Y-Y-Y-Y-Y-Y-Y-Y-Y-Yₘ-Y-Y-Y-Y-Y-Y-Zₘ-Z_{m+b}-3′
wherein X represents the RNA 5′-region, Y represents the RNA complementary region to a viral target sequence, Z represents the RNA 3′-region, m indicates a 2′-ribose modification, b indicates a phosphate backbone modification and d indicates a DNA nucleotide; and
wherein the passenger strand is subdivided into a 3′-region, and a complementary region to the guide strand, wherein the passenger strand is characterized in having the following modification pattern:
5′-Vₘ-Vₘ-V-Vₘ-Vₘ-Vₘ-V-V-V-Vₘ-Vₘ-V-Vₘ-Vₘ-Vₘ-Vₘ-V-Vₘ-Z_{m+b}-3′
wherein V represents the RNA complementary region to the guide strand, Z represents the RNA 3′-region, m indicates a 2′-ribose modification, and b indicates a phosphate backbone modification.

17. The modified siRNA according to claim 16 characterized in that one or more of the 2′-ribose modifications are selected from a group comprising 2′-O-methyl, 2′-O-methoxyethyl, 2′-deoxy-2′-fluoro, 2′-arabino-fluoro, 2′-O-benzyl and 2′-O-methyl-4-pyridine modifications.

18. The modified siRNA according to claim 16 characterized in that one or more of the phosphate backbone modifications are selected from a group comprising phosphorothioate (PS), phosphorodithioate (PS2), methylphosphonate (MP) and methoxypropylphosphonate (MOP) modifications.

19. The modified siRNA according to claim 16 characterized in that the modified siRNA targets a viral RNA sequence encoding structural or nonstructural adenoviral proteins.

20. The modified siRNA according to claim 16 characterized in that the modified siRNA targets the RNA sequence of the adenoviral proteins DNA Polymerase, pre-terminal protein, IVa2, E1A and/or the hexon protein.

21. The modified siRNA according to claim 16, wherein the siRNA is selected from the group of siRNA with one or more of the following guide strands of SEQ ID: No 39, SEQ ID: No. 41 and SEQ ID: No. 43.

22. The modified siRNA according to claim 16 encapsuled in and/or protected by lipid nanoparticles selected from the group of cationic lipids, ionizable lipids, structural lipids, and glycol-anchored lipids or is the cationic amino-lipid XL-10.

23. The modified siRNA according to claim 16 characterized in that the 5′-end is protected by and linked to a GalNAc ligand or a triantennary GalNAc ligand.

24. A pharmaceutical composition comprising the modified siRNA according to claim 16 and a pharmaceutical acceptable carrier, diluent or excipient.

25. A method of treating a viral infection and/or a viral infection caused by Adenoviridae or Mastadenoviruses comprising administering to a subject in need thereof the modified siRNA according to claim 16.

26. A method of treating an Adenoviridae related disease and/or one or more symptoms thereof in a subject, the method comprising administering to the subject the modified siRNA according to claim 16, wherein the Adenoviridae related disease and/or symptom thereof is selected from the group consisting of hepatitis, gastroenteritis, keratoconjunctivitis, cystitis, rhinitis, pharyngitis, diarrhea, respiratory diseases, obesity, and transplantation complications.


SEQUENCE LISTING (43 sequences)

SEQ ID NO: 1 - DNA, 28 bp, synthetic construct
  source 1..28: mol_type = other DNA
tcgaggctgggttatgtacttcttcttt

SEQ ID NO: 2 - DNA, 24 bp, synthetic construct
  source 1..24: mol_type = other DNA
aaagaagaagtacataacccagcc

SEQ ID NO: 3 - DNA, 28 bp, synthetic construct
  source 1..28: mol_type = other DNA
tcgaggttgggttatgtacttcttcttt

SEQ ID NO: 4 - DNA, 24 bp, synthetic construct
  source 1..24: mol_type = other DNA
aaagaagaagtacataacccaacc

SEQ ID NO: 5 - DNA, 28 bp, synthetic construct
  source 1..28: mol_type = other DNA
tcgaggctggattatgtacttcttcttt

SEQ ID NO: 6 - DNA, 24 bp, synthetic construct
  source 1..24: mol_type = other DNA
aaagaagaagtacataatccagcc

SEQ ID NO: 7 - DNA, 20 bp, synthetic construct
  source 1..20: mol_type = other DNA
cacatccaggtgcctcagaa

SEQ ID NO: 8 - DNA, 20 bp, synthetic construct
  source 1..20: mol_type = other DNA
aggtggcgtaaaggcaaatg

SEQ ID NO: 9 - DNA, 20 bp, synthetic construct
  source 1..20: mol_type = other DNA
cccctcgatgctcttagctg

SEQ ID NO: 10 - DNA, 20 bp, synthetic construct
  source 1..20: mol_type = other DNA
tcgtcttcgaacctccgact

SEQ ID NO: 11 - DNA, 21 bp, synthetic construct
  source 1..21: mol_type = other DNA
tcaacttcgccgtggacttct

SEQ ID NO: 12 - DNA, 20 bp, synthetic construct
  source 1..20: mol_type = other DNA
aggtagttgagggtggtggc

SEQ ID NO: 13 - RNA, 19 nt, Adeno-associated virus (AAV)
  source 1..19: mol_type = other RNA
gctgggttatgtacttctt

SEQ ID NO: 14 - RNA, 19 nt, Adeno-associated virus (AAV)
  source 1..19: mol_type = other RNA
gctggattatgtacttctt

SEQ ID NO: 15 - RNA, 19 nt, Adeno-associated virus (AAV)
  source 1..19: mol_type = other RNA
gctggattatgtacttctt

SEQ ID NO: 16 - RNA, 19 nt, Adeno-associated virus (AAV)
  source 1..19: mol_type = other RNA
gttgggttatgtacttctt

SEQ ID NO: 17 - RNA, 19 nt, Adeno-associated virus (AAV)
  source 1..19: mol_type = other RNA
gctggatcatgtacttctt

SEQ ID NO: 18 - RNA, 19 nt, Adeno-associated virus (AAV)
  source 1..19: mol_type = other RNA
gcgaatatgtgcaactcaa

SEQ ID NO: 19 - RNA, 19 nt, Adeno-associated virus (AAV)
  source 1..19: mol_type = other RNA
gcgaatacgtgcagctaaa

SEQ ID NO: 20 - RNA, 19 nt, Adeno-associated virus (AAV)
  source 1..19: mol_type = other RNA
gcgaatacgtgcagctaaa

SEQ ID NO: 21 - RNA, 19 nt, Adeno-associated virus (AAV)
  source 1..19: mol_type = other RNA
gcgagtacgtgcagctcaa

SEQ ID NO: 22 - RNA, 19 nt, Adeno-associated virus (AAV)
  source 1..19: mol_type = other RNA
gaaagtacgtgcagctcaa

SEQ ID NO: 23 - RNA, 19 nt, Adeno-associated virus (AAV)
  source 1..19: mol_type = other RNA
gcgagtacgtgcaactgaa

SEQ ID NO: 24 - RNA, 25 nt, Adeno-associated virus (AAV)
  source 1..25: mol_type = other RNA
caacgtcttccagcgtccaaccata

SEQ ID NO: 25 - RNA, 25 nt, Adeno-associated virus (AAV)
  source 1..25: mol_type = other RNA
taacatctttcaacggcccaccatc

SEQ ID NO: 26 - RNA, 25 nt, Adeno-associated virus (AAV)
  source 1..25: mol_type = other RNA
taacatcttccaacggcccaccatc

SEQ ID NO: 27 - RNA, 25 nt, Adeno-associated virus (AAV)
  source 1..25: mol_type = other RNA
taacgtcttccaacgccccaccatc

SEQ ID NO: 28 - DNA, 21 bp, synthetic construct
  source 1..21: mol_type = other DNA
  misc_feature 1..19: note = RNA
  misc_feature 20..21: note = DNA
ttctccgaacgtgtcacgttt

SEQ ID NO: 29 - DNA, 21 bp, synthetic construct
  source 1..21: mol_type = other DNA
  misc_feature 1..19: note = RNA
  misc_feature 20..21: note = DNA
acgtgacacgttcggagaatt

SEQ ID NO: 30 - DNA, 21 bp, synthetic construct
  source 1..21: mol_type = other DNA
  misc_feature 20..21: note = DNA
  misc_feature 1..19: note = RNA
gctgggttatgtacttctttt

SEQ ID NO: 31 - DNA, 21 bp, synthetic construct
  source 1..21: mol_type = other DNA
  misc_feature 1..19: note = RNA
  misc_feature 20..21: note = DNA
aagaagtacataacccagctt

SEQ ID NO: 32 - DNA, 21 bp, synthetic construct
  source 1..21: mol_type = other DNA
  misc_feature 1..19: note = RNA
  misc_feature 20..21: note = DNA
gcgaatacgtgcagctaaatt

SEQ ID NO: 33 - DNA, 21 bp, synthetic construct
  source 1..21: mol_type = other DNA
  misc_feature 20..21: note = DNA
  misc_feature 1..19: note = RNA
tttagctgcacgtattcgctt

SEQ ID NO: 34 - RNA, 25 nt, synthetic construct
  source 1..25: mol_type = other RNA
caacgtcttccagcgtccaaccata

SEQ ID NO: 35 - RNA, 25 nt, synthetic construct
  source 1..25: mol_type = other RNA
tatggttggacgctggaagacgttg

SEQ ID NO: 36 - RNA, 19 nt, synthetic construct
  source 1..19: mol_type = other RNA
  modified_base 14: mod_base = OTHER; note = 2'-O-methylribose nucleotide
  modified_base 15: mod_base = OTHER; note = 2'-O-methylribose nucleotide
  modified_base 4: mod_base = OTHER; note = 2'-O-methylribose nucleotide
  modified_base 2: mod_base = OTHER; note = 2'-O-methylribose nucleotide
  modified_base 16: mod_base = OTHER; note = 2'-O-methylribose nucleotide
  modified_base 5: mod_base = OTHER; note = 2'-O-methylribose nucleotide
  modified_base 18: mod_base = OTHER; note = 2'-O-methylribose nucleotide
  modified_base 19: mod_base = OTHER; note = phosphorothioate nucleotide
  modified_base 1: mod_base = OTHER; note = 2'-O-methylribose nucleotide
  modified_base 11: mod_base = OTHER; note = 2'-O-methylribose nucleotide
  modified_base 19: mod_base = OTHER; note = 2'-O-methylribose nucleotide
  modified_base 6: mod_base = OTHER; note = 2'-O-methylribose nucleotide
  modified_base 13: mod_base = OTHER; note = 2'-O-methylribose nucleotide
  modified_base 10: mod_base = OTHER; note = 2'-O-methylribose nucleotide
ttctccgaacgtgtcacgt

SEQ ID NO: 37 - DNA, 21 bp, synthetic construct
  source 1..21: mol_type = other DNA
  misc_difference 2..21: note = RNA
  misc_feature 1: note = DNA
  modified_base 21: mod_base = OTHER; note = 2'-O-methylribose nucleotide
  modified_base 21: mod_base = OTHER; note = phosphorothioate nucleotide
  modified_base 20: mod_base = OTHER; note = 2'-O-methylribose nucleotide
  modified_base 13: mod_base = OTHER; note = 2'-O-methylribose nucleotide
acgtgacacgttcggagaatt

SEQ ID NO: 38 - RNA, 19 nt, synthetic construct
  source 1..19: mol_type = other RNA
  modified_base 14: mod_base = OTHER; note = 2'-O-methylribose nucleotide
  modified_base 10: mod_base = OTHER; note = 2'-O-methylribose nucleotide
  modified_base 13: mod_base = OTHER; note = 2'-O-methylribose nucleotide
  modified_base 5: mod_base = OTHER; note = 2'-O-methylribose nucleotide
  modified_base 11: mod_base = OTHER; note = 2'-O-methylribose nucleotide
  modified_base 19: mod_base = OTHER; note = phosphorothioate nucleotide
  modified_base 19: mod_base = OTHER; note = 2'-O-methylribose nucleotide
  modified_base 4: mod_base = OTHER; note = 2'-O-methylribose nucleotide
  modified_base 15: mod_base = OTHER; note = 2'-O-methylribose nucleotide
  modified_base 6: mod_base = OTHER; note = 2'-O-methylribose nucleotide
  modified_base 16: mod_base = OTHER; note = 2'-O-methylribose nucleotide
  modified_base 18: mod_base = OTHER; note = 2'-O-methylribose nucleotide
  modified_base 1: mod_base = OTHER; note = 2'-O-methylribose nucleotide
  modified_base 2: mod_base = OTHER; note = 2'-O-methylribose nucleotide
gctgggttatgtacttcta

SEQ ID NO: 39 - DNA, 21 bp, synthetic construct
  source 1..21: mol_type = other DNA
  misc_feature 2..21: note = RNA
  misc_feature 1: note = DNA
  modified_base 13: mod_base = OTHER; note = 2'-O-methylribose nucleotide
  modified_base 21: mod_base = OTHER; note = 2'-O-methylribose nucleotide
  modified_base 21: mod_base = OTHER; note = phosphorothioate nucleotide
  modified_base 20: mod_base = OTHER; note = 2'-O-methylribose nucleotide
tagaagtacataacccagctt

SEQ ID NO: 40 - RNA, 19 nt, synthetic construct
  source 1..19: mol_type = other RNA
  modified_base 10: mod_base = OTHER; note = 2'-O-methylribose nucleotide
  modified_base 13: mod_base = OTHER; note = 2'-O-methylribose nucleotide
  modified_base 15: mod_base = OTHER; note = 2'-O-methylribose nucleotide
  modified_base 18: mod_base = OTHER; note = 2'-O-methylribose nucleotide
  modified_base 5: mod_base = OTHER; note = 2'-O-methylribose nucleotide
  modified_base 16: mod_base = OTHER; note = 2'-O-methylribose nucleotide
  modified_base 19: mod_base = OTHER; note = 2'-O-methylribose nucleotide
  modified_base 6: mod_base = OTHER; note = 2'-O-methylribose nucleotide
  modified_base 14: mod_base = OTHER; note = 2'-O-methylribose nucleotide
  modified_base 2: mod_base = OTHER; note = 2'-O-methylribose nucleotide
  modified_base 11: mod_base = OTHER; note = 2'-O-methylribose nucleotide
  modified_base 4: mod_base = OTHER; note = 2'-O-methylribose nucleotide
  modified_base 1: mod_base = OTHER; note = 2'-O-methylribose nucleotide
  modified_base 19: mod_base = OTHER; note = phosphorothioate nucleotide
gcgaatacgtgcagctaaa

SEQ ID NO: 41 - DNA, 21 bp, synthetic construct
  source 1..21: mol_type = other DNA
  misc_feature 2..21: note = RNA
  misc_feature 1: note = DNA
  modified_base 20: mod_base = OTHER; note = 2'-O-methylribose nucleotide
  modified_base 21: mod_base = OTHER; note = phosphorothioate nucleotide
  modified_base 13: mod_base = OTHER; note = 2'-O-methylribose nucleotide
  modified_base 21: mod_base = OTHER; note = 2'-O-methylribose nucleotide
tttagctgcacgtattcgctt

SEQ ID NO: 42 - RNA, 19 nt, synthetic construct
  source 1..19: mol_type = other RNA
  modified_base 1: mod_base = OTHER; note = 2'-O-methylribose nucleotide
  modified_base 13: mod_base = OTHER; note = 2'-O-methylribose nucleotide
  modified_base 11: mod_base = OTHER; note = 2'-O-methylribose nucleotide
  modified_base 4: mod_base = OTHER; note = 2'-O-methylribose nucleotide
  modified_base 14: mod_base = OTHER; note = 2'-O-methylribose nucleotide
  modified_base 15: mod_base = OTHER; note = 2'-O-methylribose nucleotide
  modified_base 5: mod_base = OTHER; note = 2'-O-methylribose nucleotide
  modified_base 10: mod_base = OTHER; note = 2'-O-methylribose nucleotide
  modified_base 19: mod_base = OTHER; note = 2'-O-methylribose nucleotide
  modified_base 2: mod_base = OTHER; note = 2'-O-methylribose nucleotide
  modified_base 18: mod_base = OTHER; note = 2'-O-methylribose nucleotide
  modified_base 19: mod_base = OTHER; note = phosphorothioate nucleotide
  modified_base 16: mod_base = OTHER; note = 2'-O-methylribose nucleotide
  modified_base 6: mod_base = OTHER; note = 2'-O-methylribose nucleotide
caacgtcttccagcgtcca

SEQ ID NO: 43 - DNA, 21 bp, synthetic construct
  source 1..21: mol_type = other DNA
  misc_feature 1: note = DNA
  misc_feature 2..21: note = RNA
  modified_base 21: mod_base = OTHER; note = 2'-O-methylribose nucleotide
  modified_base 13: mod_base = OTHER; note = 2'-O-methylribose nucleotide
  modified_base 21: mod_base = OTHER; note = phosphorothioate nucleotide
  modified_base 20: mod_base = OTHER; note = 2'-O-methylribose nucleotide
tggacgctggaagacgttgtt